(19) United States
(12) Patent Application Publication
(10) Pub. No.: US 2025/0352637 A1
(43) Pub. Date: Nov. 20, 2025

(54) METHOD FOR SUSTAINED DELIVERY OF MRNA VACCINES

(71) Applicant: Merck Sharp & Dohme LLC, Rahway, NJ (US)

(72) Inventors: Andrew Bett, Lansdale, PA (US); Marian E. Gindy, Rahway, NJ (US)

(73) Assignee: Merck Sharp & Dohme LLC, Rahway, NJ (US)

(21) Appl. No.: 18/871,320

(22) PCT Filed: Jun. 2, 2023
(86) PCT No.: PCT/US2023/024220

§ 371 Date: Dec. 3, 2024

Related U.S. Application Data
(60) Provisional application No. 63/350,071, filed on Jun. 8, 2022.

(51) Int. Cl. A61K 39/155; A61K 9/00; A61K 31/7105; A61K 39/00; A61P 31/00

(52) U.S. Cl. CPC A61K 39/155; A61K 9/0021; A61K 31/7105; A61P 31/00; A61K 2039/53; A61K 2039/545; A61K 2039/55555; A61K 2039/6018

(57) ABSTRACT

The invention relates to a method of treating a disease or disorder in a patient in need thereof that includes providing an active pharmaceutical ingredient (API) to the patient by administering more than one split-dose of the API over a pre-determined period of time. In embodiments of the invention, the API is an mRNA encoding an antigen. The attractiveness of mRNA as a vaccine modality is supported by several advantages. As a non-infectious agent that does not require incorporation into the host's genome to confer activity along with its well-defined chemical composition, mRNA is regarded as a relatively safe vaccine modality.

### CROSS-REFERENCE TO RELATED APPLICATIONS

This application claims the benefit of priority to U.S. Provisional Application No. 63/350,071 filed Jun. 8, 2022, the disclosure of which is incorporated herein by its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 19, 2022, is named 25402_WO_PCT_SL.XML and is 5,819 bytes in size.

### FIELD OF THE INVENTION

The invention relates to a method of treating a disease or disorder in a patient in need thereof that includes providing an active pharmaceutical ingredient (API) to the patient by administering more than one split-dose of the API over a pre-determined period of time. In embodiments of the invention, the API is an mRNA encoding an antigen.

### BACKGROUND OF THE INVENTION

Vaccination is regarded as one of the greatest successes in modern medicine. (See Plotkin, S. L. & Plotkin, S. A. in Vaccines (Sixth Edition) (eds Stanley A. Plotkin, Walter A. Orenstein, & Paul A. Offit) 1-13 (W.B. Saunders, 2013).) Concerted vaccination efforts have largely eradicated smallpox, measles and polio and significantly contributed to reducing the burden of many other transmittable infectious diseases. Conventional vaccine approaches, including live-attenuated and inactivated viruses, protein carrier conjugates and subunit protein/adjuvant combinations, have been shown to elicit robust immune responses and provide durable protection against these communicable diseases. (See Plotkin, S. L. & Plotkin, S. A. in Vaccines (Sixth Edition) (eds Stanley A. Plotkin, Walter A. Orenstein, & Paul A. Offit) 1-13 (W.B. Saunders, 2013); Thomas, S. Vaccine Design-Methods and Protocols Volume 1: Vaccines for Human Diseases. (Springer-Humana Press, 2016). Despite the successes of these approaches, there remains a need to develop next-generation vaccines that not only drive the necessary immune responses but can also be more rapidly produced to facilitate clinical and industrial translation. (See Rauch, S., Jasny, E., Schmidt, K. E. & Petsch, B. New Vaccine Technologies to Combat Outbreak Situations. Front. Immunol. 9, doi: 10.3389/fimmu.2018.01963 (2018); van Riel, D. & de Wit, E. Next-generation vaccine platforms for COVID-19. Nat. Mater. 19, 810-812, doi: 10.1038/s41563-020-0746-0 (2020). DeFrancesco, L. The ‘anti-hype’ vaccine. Nat. Biotechnol. 35, 193-197, doi: 10.1038/nbt.3812 (2017).)

mRNA vaccines have emerged as a leading next-generation vaccine approach driven primarily by the recent emergency use authorization of Pfizer-BioNTech (BNT162b2) and Moderna (mRNA-1273) mRNA vaccines for the prevention of coronavirus disease 2019 (COVID-19) caused by severe acute respiratory syndrome coronavirus 2 (SARS-COV-2). (See Baden, L. R. et al. Efficacy and Safety of the mRNA-1273 SARS-COV-2 Vaccine. N. Engl. J. Med. 384, 403-416, doi: 10.1056/NEJMoa2035389 (2020); Polack, F. P. et al. Safety and Efficacy of the BNT162b2 mRNA Covid-19 Vaccine. N. Engl. J. Med. 383, 2603-2615, doi: 10.1056/NEJMoa2034577 (2020).) Since the initial studies that showed in vitro transcribed mRNA could produce protein and cause a pharmacodynamic response in mice, there have been significant advancements in the field. (See Wolff, J. et al. Direct gene transfer into mouse muscle in vivo. Science 247, 1465-1468, doi: 10.1126/science. 1690918 (1990); Jirikowski, G., Sanna, P., Maciejewski-Lenoir, D. & Bloom, F. Reversal of diabetes insipidus in Brattleboro rats: intrahypothalamic injection of vasopressin mRNA. Science 255, 996-998, doi: 10.1126/science.1546298 (1992).) mRNA sequence optimization, the incorporation of modified nucleosides and codon optimization were critical to improving instability against enzymatic degradation and mitigating recognition by innate immune receptors that diminished mRNA translation. Further, advances in RNA formulation and use of specialized carrier systems have allowed for more efficient intracellular delivery of mRNA, resulting in improved expression and presentation of translated antigens upon in vivo administration. (See Huang, L. et al. Current Topics in Microbiology and Immunology Ch. Chapter 222, (2020); Kowalski, P. S., Rudra, A., Miao, L. & Anderson, D. G. Delivering the Messenger: Advances in Technologies for Therapeutic mRNA Delivery. Mol. Ther. 27, 710-728, doi: 10.1016/j.ymthe.2019.02.012 (2019); Zeng, C., Zhang, C., Walker, P. G. & Dong, Y. Formulation and Delivery Technologies for mRNA Vaccines. (Springer Berlin Heidelberg)). Delivery carriers, such as lipid-derived and polymer-derived materials, previously used to deliver small molecule drugs and siRNAs, have been adapted for mRNA delivery. Of these systems, lipid nanoparticles (LNPs) are the most clinically advanced and serve as components of both the Pfizer-BioNTech and Moderna COVID-19 vaccines.

The attractiveness of mRNA as a vaccine modality is supported by several advantages. As a non-infectious agent that does not require incorporation into the host's genome to confer activity along with its well-defined chemical composition, mRNA is regarded as a relatively safe vaccine modality. mRNA can also enable conformationally-driven immune responses as it undergoes endogenous translation to the target protein antigen. This is a particular advantage for antigens whose immunogenic conformation is difficult to stabilize through traditional in vitro subunit protein production, such as pre-fusion RSV-F. Additionally, the ability to enable rapid immunogen discovery and faster manufacturing relative to traditional vaccine approaches offers significant promise in enabling accelerated deployment of new vaccines.

Despite these advantages, there remain opportunities for improvements of mRNA vaccines. A primary hurdle for efficient access and deployment of mRNA vaccines on a global scale is the vaccine supply chain. The development of cost-effective vaccine regimens, vaccines with improved temperature stability and those requiring less-frequent dosing are necessary to make mRNA vaccines more practical and affordable for a greater number of people in countries across the world. mRNA is a highly customized component which has limited the availability of supply produced consistent with good manufacturing practice (GMP) and that meets the required purity and potency quality attributes. This specialization in production and limited GMP manufacturing access currently result in a high cost-of-goods of the mRNA, particularly when compared to more traditional vaccine platforms. (See Kis, Z., Kontoravdi, C., Shattock, R. & Shah, N. Resources, Production Scales and Time Required for Producing RNA Vaccines for the Global Pandemic Demand. Vaccines 9, doi: 10.3390/vaccines9010003 (2020); Kis, Z., Kontoravdi, C., Dey, A. K., Shattock, R. & Shah, N. Rapid development and deployment of high-volume vaccines for pandemic response. J. Adv. Manuf. Process. 2, e10060 (2020).) Production methods that improve the efficiency of mRNA manufacture or vaccine technologies that lower the mRNA dose are thus required to reduce the cost-of-goods and enable the viability of mRNA vaccines on a global scale.

mRNA has emerged as a promising modality for next-generation vaccines as it has been shown to elicit strong humoral and cellular immune responses, is considered to have an acceptable safety profile and can be rapidly developed. Despite their potential, industrial challenges have limited realization of the vaccine platform on a global scale. Critical among these challenges are supply chain considerations, including mRNA production, cost of goods and vaccine frozen-chain distribution. There is a need to investigate alternative mRNA vaccine dose regimens, formulations, and/or vaccine delivery strategies that could reduce the overall mRNA dose required while still maintaining the necessary vaccine efficacy.

### SUMMARY OF THE INVENTION

The present invention provides a method of treating a disease or disorder in a patient in need thereof comprising: providing an active pharmaceutical ingredient (API) to said patient comprising: (a) administering a first split-dose of said API; (b) waiting for a pre-determined amount of time to pass; (c) administering an additional split-dose of said API; and optionally repeating steps (b) and (c); wherein each split-dose comprises an amount of API that is less than the amount of said API that is determined to be effective at treating said disease or disorder via a bolus dose.

In one embodiment, the API is provided to the patient as an mRNA composition comprising an mRNA encoding an antigen and a pharmaceutically acceptable carrier. In one embodiment, the mRNA composition further comprises a lipid nanoparticle (LNP). In one embodiment, the LNP comprises a cationic lipid, a phospholipid, cholesterol, and a PEG-lipid. In one embodiment, the LNP comprises 30-65 mole % cationic lipid, 5-30 mole % phospholipid, 10-40 mole % cholesterol, and 0.5-4 mole % PEG-lipid. In one embodiment, the LNP comprises DSPC, cholesterol, ePEG2000-DMG, and (13Z, 16Z)-N, N-dimethyl-3-nonyldocosa 13, 16-dien-1-amine.

In one embodiment, the total amount of API provided to the patient by administration of all split-doses is equal to X % of the amount of the API provided in a bolus dose of said API, wherein X is less than or equal to 100. In one embodiment, the amount of API in each split-dose is the same. In one embodiment, the amount of API in each split-dose is not the same. In some embodiments, one or more of the split doses does not have the same amount of API. For example, if there are 3 split-doses, 2 can be the same and 1 can be different or every dose can be a different amount of API or if there are 5 split does, 4 can be the same and one can be different, or 3 can include the same amount of API and the remaining 2 can be the same amount of API or different, or all 5 split-doses can all be different.

In one embodiment, the therapeutic effect is the same or greater than such effect when said API is provided as a bolus dose. In one embodiment, the ΔT_{1/2} of the API provided as a split-dose is greater than the ΔT_{1/2} when the API is provided as a bolus dose. In one embodiment, the ΔT_{1/2} of the API provided as a split-dose is at least 2-10 times greater than the ΔT_{1/2} when the API is provided as a bolus dose. In one embodiment, the Rₘₐₓ of the API provided as a split-dose is less than the Rₘₐₓ when the API is provided as a bolus dose. In one embodiment, the Rₘₐₓ of the API provided as a split-dose is at least 50% less than the Rₘₐₓ when the API is provided as a bolus dose. In one embodiment, the AUC of the API provided as a split-dose is approximately the same as the AUC when the API is provided as a bolus dose.

In one embodiment, a method of inducing an immune response in a patient in need thereof is provided comprising: providing an active pharmaceutical ingredient (API) to said patient comprising: administering a first split-dose of said API; waiting for a pre-determined amount of time to pass; administering an additional split-dose of said API; and optionally repeating steps (b) and (c); wherein each split-dose comprises an amount of API that is less than the amount of said API that is determined to be therapeutically effective at inducing an immune response via a bolus dose.

### BRIEF DESCRIPTION OF THE DRAWINGS

FIG. 1 depicts protein expression kinetics following administration of a SEAP mRNA/LNP vaccine in BALB/c mice at 1 μg and 10 μg mRNA dose, as described in Example 4. Peak SEAP protein levels in serum were achieved within 24 hours and steadily declined over the course of 7 days. Data are expressed as the geometric mean with error bars representing the 95% confidence interval. Statistical significance is denoted as **p<0.01.

FIGS. 2A-2D depict the results of the population PKPD model and simulation results described in Example 5. FIG. 2A depicts mRNA split-dose schedules used to model SEAP protein expression. FIG. 2B is a schematic diagram of PKPD model and related key differential equations. FIG. 2C depicts simulated response curves under different API release schedules (1 μg treatment). FIG. 2D is a schematic of the key parameters in calculating ΔT_{1/2}.

FIGS. 3A-3D depict the results of the split-dose administration of an RSV pre-F mRNA/LNP vaccine compared to bolus administration of the same vaccine, as shown in Example 6. FIG. 3A depicts the immunization schedules of an RSV pre-F mRNA/LNP vaccine administered to BALB/c mice at 0.1 μg and 0.5 μg mRNA doses in bolus and split-dose regimens; cumulative RSV pre-F mRNA dose administered in split-dose regimen was studied; FIGS. 3B and 3C depict total IgG response to RSV F determined by ELISA at day 21 (FIG. 3B) and day 42 (FIG. 3C), comparing split-dose and bolus dose regimens. FIG. 3D depicts RSV serum neutralizing titers comparing split-dose and bolus dose regimens. Data are expressed as the geometric mean with error bar representing the 95% confidence interval. Dotted line indicates assay limit of detection. Statistical significance is denoted as *p<0.05 and **p<0.01.

FIGS. 4A-4D show that the split-dose administration of an unadjuvanted DS-Cav1 subunit protein vaccine elicited an inferior immune response compared to bolus administration of the same vaccine, as described in Example 7. FIG. 4A depicts immunization schedules of the unadjuvanted DS-Cav1 vaccine administered to BALB/c mice at 0.1 μg doses in bolus and split-dose regimens; cumulative DS-Cav1 dose administered in split-dose regimen was also studied. FIGS. 4B and 4 C depict total IgG responses to an RSV pre-F vaccine determined by ELISA at days 21 and 42, respectively, comparing split-dose and bolus dose regimens of the same vaccine. FIG. 4D depicts RSV serum neutralizing titers comparing split-dose and bolus dose regimens. Data are expressed as the geometric mean with error bars representing the 95% confidence interval. Dotted line indicates assay limit of detection. Statistical significance is denoted as *p<0.05 and **p<0.01.

FIGS. 5A-5C shows that split-dose administration of adjuvanted DS-Cav1 subunit protein elicits improved immune response compared to traditional bolus administration, as described in Example 7. Immunization schedules and cumulative DS-Cav1 dose administered in split-dose regimen studied were as shown in FIG. 3A. FIGS. 5A and 5B depict total IgG responses to RSV pre-F determined by ELISA at days 21 and 42, respectively, comparing split-dose and bolus dose administration. FIG. 5C depicts RSV serum neutralizing titers comparing split-dose and bolus dose administration. Data are expressed as the geometric mean with error bars representing the 95% confidence interval. Dotted line indicates assay limit of detection. Statistical significance is denoted as *p<0.05 and **p<0.01.

FIGS. 6A-6C shows that RSV mRNA/LNP vaccine delivered following additional split-dose regimen shown in FIG. 3A resulted in improved humoral immunity compared to traditional bolus administration, as described in Example 7. FIGS. 6A and 6B depict total IgG responses to RSV pre-F determined by ELISA at day 21 and 42, respectively, comparing split-dose vs bolus dose administration. FIG. 6C depicts RSV serum neutralizing titers comparing split-dose vs bolus dose regimens. Data are expressed as the geometric mean with error bars representing the 95% confidence interval. Dotted line indicates assay limit of detection. Statistical significance is denoted as *p<0.05 and **p<0.01.

### DETAILED DESCRIPTION OF THE INVENTION

### Abbreviations and Definitions

As used throughout the specification and appended claims, the following abbreviations apply:
API active pharmaceutical ingredient
AUC area under the curve
CHO Chinese hamster ovary
DMG dimyristoyl glycerol
DSPC distearoylphosphatidylcholine
ELISA enzyme-linked immunosorbant assay
IgG immunoglobulin G
LNP lipid nanoparticle
mRNA messenger RNA
NONMEM nonlinear mixed effects modeling
PEG poly(ethyleneglycol)
PKPD pharmacokinetic/pharmacodynamic
RSV respiratory syncytial virus
SEAP secreted embryonic alkaline phosphatase
v/v volume per volume
WFI water for injection
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise. Reference to “or” indicates either or both possibilities unless the context clearly dictates one of the indicated possibilities. In some cases, “and/or” was employed to highlight either or both possibilities.

About: The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1%, ±0.5%, ±1%, ±2%, ±3%, ±4%, ±5%, ±6%, ±7%, ±8%, ±9%, ±10% or ±11%. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Adjuvant: As used herein, the term “adjuvant” refers to a composition or compound that is capable of enhancing the immune response against an antigen of interest. Adjuvants are substances or combinations of substances that are used in conjunction with a vaccine antigen to enhance (e.g., increase, accelerate, prolong and/or possibly target) the specific immune response to the vaccine antigen or modulate to a different type (e.g., switch a Th1 immune response to a Th2 response, or a humoral response to a cytotoxic T cell response) in order to enhance the clinical effectiveness of the vaccine. In some embodiments, the adjuvant modifies (Th1/Th2) the immune response. In some embodiments, the adjuvant boosts the strength and longevity of the immune response. In some embodiments, the adjuvant broadens the immune response to a concomitantly administered antigen. In some embodiments, the adjuvant is capable of inducing strong antibody and T cell responses. In some embodiments, the adjuvant is capable of increasing the polyclonal ability of the induced antibodies. In some embodiments, the adjuvant is used to decrease the amount of antigen necessary to provoke the desired immune response and provide protection against the disease. In some embodiments, the adjuvant is used to decrease the number of injections needed in a clinical regimen to induce a durable immune response and provide protection against the disease. Adjuvant containing formulations described herein may demonstrate enhancements in humoral and/or cellular immunogenicity of vaccine antigens, for example, subunit vaccine antigens.

Administration: As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a VLP, a small molecule, polypeptide (e.g., a protein), polynucleotide (e.g., a DNA polynucleotide or an RNA polynucleotide), saccharide, lipid, or a combination or complex thereof. In some embodiments, the term “agent” can refer to a compound, molecule, or entity that includes a polymer, or a plurality thereof.

Alkyl and Alkenyl: As used herein, the term “alkyl” refers to a straight chain, cyclic or branched saturated aliphatic hydrocarbon having the specified number of carbon atoms. A numerical range, which refers to the chain length in total, may be given. For example, C₁-C₆ heteroalkyl has a chain length of 1 to 6 atoms. As used herein, the term “alkenyl” means a straight chain, cyclic or branched unsaturated aliphatic hydrocarbon having the specified number of carbon atoms including but not limited to diene, triene and tetraene unsaturated aliphatic hydrocarbons.

Antibody: As used herein, the term “antibody” (or “Ab”) refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies.

Antigen: As used herein, the term “antigen” refers to any antigen that can generate one or more immune responses. The antigen may be a protein (including recombinant proteins), VLP, polypeptide, or peptide (including synthetic peptides). The antigen may be one that generates a humoral and/or CTL immune response.

API: As used herein, the term “API” refers to an active pharmaceutical ingredient, drug, or compound, which, in some embodiments, is a component of a composition or formulation as disclosed herein that is biologically active (e.g. capable of inducing an appropriate immune response) and confers a therapeutic or prophylactic benefit to a person or animal in need thereof. As used herein, an API may be a vaccine active ingredient such as a nucleic acid molecule such as mRNA that encodes an antigen, which can induce an immune response when administered to a patient.

Aryl: As used herein, the term “aryl” refers to a carbocycle aromatic monocyclic or bicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is phenyl. Unless otherwise indicated, an aryl group is unsubstituted.

AUC: As used herein, the term “AUC” refers to the area under the plasma API concentration-time curve. The AUC reflects the actual body exposure to the API after administration of a dose of the pharmaceutical composition, which includes the API. The AUC is dependent on the rate of elimination of the drug from the body and the dose administered. In some instances, the pharmaceutical composition may be a vaccine and the API may be an mRNA-expressed protein (e.g. SEAP) and the plasma AUC of the expressed SEAP is measured as a function of time (ng/ml). The AUC is dependent on the rate of elimination of the expressed protein and the dose administered.

Bolus: As used herein, the term “bolus” or “bolus dose” refers to the single administration of a discrete amount of API. In some embodiments, a bolus dose comprises an amount of API that is determined through experimentation (e.g. a clinical trial) or expected to be effective at bringing about a desired therapeutic effect such as decreasing or eliminating a disease or disorder or ameliorating the symptoms thereof, inducing an immune response against an antigen, inducing a protective immune response against an antigen, decreasing the likelihood of infection, or decreasing the number or severity of symptoms of a disease or disorder; wherein such amount is administered to a patient in a single administration. The term “bolus dose” does not mean that additional administrations of an amount of said API may not be given as part of a therapeutic treatment regimen. For example, a bolus dose of 100 μg of protein X may be administered as a vaccine to a patient to induce an immune response against protein X and 50 μg of protein X may be administered to the patient several months later to boost the immune response. Both the 100 μg dose and the 50 μg dose would be considered bolus doses which individually bring about a desired therapeutic effect and also bring about a therapeutic effect collectively as part of the treatment regimen. In some embodiments, the bolus dose could refer to a therapeutically effective amount.

Comprising or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

ΔT_{1/2}: As used herein, the term “ΔT_{1/2}” means the duration of time reflecting elevated protein expression. The term is further defined by the following formula:

$$Δ {T}_{1 / 2} = {T}_{end} - {T}_{start} .$$

Cationic lipid: As used herein, the term “cationic lipid” refers to a lipid species that carries a net positive charge at a selected pH, such as physiological pH. Those of skill in the art will appreciate that a cationic lipid can include, but are not limited to, U.S. Patent Application Publication Nos. US 2008/0085870, US 2008/0057080, US 2009/0263407, US 2009/0285881, US 2010/0055168, US 2010/0055169, US 2010/0063135, US 2010/0076055, US 2010/0099738, US 2010/0104629, US 2013/0017239, and US 2016/0361411, International Patent Application Publication Nos. WO2011/022460; WO2012/040184, WO2011/076807, WO2010/021865, WO2009/132131, WO2010/042877, WO2010/146740, WO2010/105209, and in U.S. Pat. Nos. 5,208,036, 5,264,618, 5,279,833, 5,283,185, 6,890,557, and 9,669,097.

Dose: As used herein, the term “dose” means a quantity of an agent, API, formulation, or pharmaceutical composition administered or recommended to be administered at a particular time or over a particular time period to provide a desired therapeutic or prophylactic effect. For example, in some instances, a dose may be a single administration of a specific quantity of a pharmaceutical composition. In other embodiments, a dose may be multiple administrations of a specific quantity of a pharmaceutical composition.

Heteroalkyl: As used herein, the term “heteroalkyl” refers to an alkyl moiety as defined above, having one or more carbon atoms, for example one, two or three carbon atoms, replaced with one or more heteroatoms, which may be the same or different, where the point of attachment to the remainder of the molecule is through a carbon atom of the heteroalkyl radical. The heteroalkyl groups may be substituted. Unless otherwise stated in the specification, heteroalkyl groups may be substituted at carbon atoms in the radicals with one or more substituents which independently are oxo, fluoro, C₁-C₃ alkyl, C₁-C₃ fluoroalkyl, amino, or hydroxy. In some embodiments, the heteroalkyl groups have 1-2 heteroatoms selected from nitrogen, sulfur and oxygen atoms in the atom chain. In some embodiments, the heteroalkyl groups have 1 heteroatom selected from nitrogen, sulfur and oxygen atoms in the atom chain. In some embodiments, the heteroatoms are selected from O, S, S(O), S(O)₂, and —NH—, —N(alkyl)-. Non-limiting examples include ethers, thioethers, amines, hydroxymethyl, 3-hydroxypropyl, 1,2-dihydroxyethyl, 2-methoxyethyl, 2-aminoethyl, 2-dimethylaminoethyl, and the like an aliphatic group containing a heteroatom.

Heteroaryl: As used herein, the term “heteroaryl” refers to means an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

Immunogenicity: As used herein, the term “immunogenicity” relates to the relative effectivity of an antigen to induce an immune reaction.

K_{IN} and K_{OUT}: As used herein, the terms “K_{IN}” and “K_{OUT}” refer to biomarker production rate and elimination rate constant independent of feedback. In one embodiment, K_{IN} can be considered the rate at which the API or drug binds to the receptor. In one embodiment, K_{OUT} refers to the rate constant for API or drug disassociation from the receptor.

Lipid: As used herein, the term “lipid” refers to any of a group of organic compounds that are esters of fatty acids and are characterized by being insoluble in water or having low solubility in water but may be soluble in many organic solvents.

Lipid nanoparticle: As used herein, the term “lipid nanoparticle” (or “LNP”) refers to a lipid that forms a particle having a length or width measurement (e.g., a maximum length or width measurement) between 10 and 1000 nanometers.

mRNA: The term “mRNA” means “messenger-RNA” and relates to a “transcript” which is generated by using a DNA template and encodes a peptide or protein. Typically, an mRNA comprises a 5′-UTR, a protein coding region and a 3′-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available. In the context of the present invention, the RNA, preferably the mRNA, is modified with a 5′-cap structure.

NONMEM: As used herein, the term “NONMEM” refers to nonlinear mixed effects modeling. In one embodiment, the specific NONMEM is PsN 4.7.15.

Patient: As used herein, the term “patient” (alternatively referred to as “subject” or “individual” herein) refers to an organism, typically a mammal (e.g., rat, mouse, dog, cat, rabbit, human, in some embodiments including prenatal human forms) capable of being treated with the methods and compositions of the invention, most preferably a human. In some embodiments, the patient is an adult patient. In other embodiments, the patient is a pediatric patient. A patient “in need of treatment” means that the subject has been identified as having a need for the particular method or treatment. In some embodiments, a patient displays one or more symptoms or characteristics of a disease, disorder or condition. In some embodiments, a patient does not display any symptom or characteristic of a disease, disorder, or condition. In some embodiments, the identification can be by any means of diagnosis. In some embodiments, the patient is in an environment or will be traveling to an environment in which a particular disease, disorder, or condition is prevalent. In some embodiments, the patient is at risk of developing a particular disease or disorder that a treatment is intended to treat and/or prevent. Those “in need of treatment” include those patients that may benefit from treatment with the methods of the inventions, e.g. a patient suffering from or at risk of developing a disease or disorder. In some embodiments, a patient is someone with one or more features characteristic of susceptibility to or risk of a disease, disorder, or condition. In some embodiments, a patient is an individual to whom diagnosis and/or therapy is and/or has been administered.

Pharmaceutical composition: As used herein, the term “pharmaceutical composition,” refers to a composition containing an active pharmaceutical or biological ingredient, along with one or more additional components, e.g. a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, the term formulation refers to a single-dose of vaccine, which can be included in any volume suitable for injection.

Pharmaceutically acceptable: As used herein, the term “pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Pharmacokinetics: As used herein, the term “pharmacokinetics” refers to the absorption, distribution, metabolism, and elimination of APIs from the body.

Pharmacodynamics: As used herein, the term “pharmacodynamics” refers to the interaction of APIs with target tissues.

Pharmacokinetic steady state: As used herein, the term “pharmacokinetic steady state” or “steady state” refers to a period of time during which any accumulation of API concentrations owing to multiple doses has been maximized and systemic API exposure is considered uniform after each subsequent dose administered.

Rₘₐₓ: As used herein, the term “Rₘₐₓ” or “Cₘₐₓ” refers to the maximum plasma API concentration in the AUC curve. The Rₘₐₓ or Cₘₐₓ is the peak plasma concentration of an API after dosing.

Split-Dose: As used herein, the term “split-dose” refers to an administration of a discrete amount of API to a patient wherein the amount of API is less than the amount that was previously determined, expected, or hypothesized to bring about a desired therapeutic effect via a bolus dose, but more than one such split-dose is administered to the patient over a pre-determined amount of time. For example, a bolus dose of 100 μg of compound Y may instead be provided to a patient as 3, 4, 5, or 6 split-doses of 25, 50, or 75 μg each over a period of time such as two weeks. In this example, each split dose may be provided to a patient over equal periods, e.g., every ½ week for 2 weeks, or may be provided over unequal periods, e.g. the first split-dose at day 0, the second split-dose at day 3, the third split dose at day 10 and the fourth split-dose at day 14. In embodiments of the invention, the administration of more than one split-dose of an API to a patient over a pre-determined amount of time results in a therapeutic effect that is the same as or greater than the therapeutic effect that would result from administering such API to the patient as a single bolus dose, e.g., in some embodiments, a regimen of a vaccine comprising an mRNA encoding an antigen provided to a patient as several split-doses over a pre-determined amount of time results in an enhanced immune response as the same mRNA vaccine given to the patient as a single bolus dose.

Statistical Analyses/Significance: As used herein, statistical significance or statistical analyses was determined using unpaired, two-tailed Student's T-test using GraphPad Prism 9.0 software (GraphPad, San Diego, CA, USA). Data are expressed as the geometric mean with error bars representing the 95% confidence interval. Differences were considered statistical significance at *p<0.05 and **p<0.01.

Sustained Delivery: As used herein, the term “sustained delivery” refers to the introduction of a discrete amount of an API in the body by controlling the rate or time of delivery to the patient, e.g. through the administration of more than one split-dose of the API or through the administration of more than one split-dose of a composition comprising the API over a pre-determined period of time.

Sustained Release: As used herein, “sustained release” or “controlled release” refers to the rate at which an API is released from a pharmaceutical composition as a function of time.

Secreted embryonic alkaline phosphatase (“SEAP”): As used herein, the term “SEAP” refers to secreted embryonic alkaline phosphatase protein that is a truncated form of human placental alkaline phosphatase that comprises 520 amino acids (SEQ ID NO. 1). Those of skill in the art will appreciate that SEAP is expressed in CHO cells and shows a 75 kDa band on SDS page. Recombinant SEAP protein is purified by affinity chromatography.

Therapeutically Effective Amount: As used herein, the term “therapeutically effective amount” or “therapeutically effective” refers to an amount of the active ingredient (e.g. therapeutic protein, vaccine, or antibody) sufficient to produce the desired therapeutic effect in a human or animal, e.g. the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given antibody or therapeutic protein or vaccine antigen. A therapeutically effective amount of an active ingredient may be provided to a patient by a single delivery or more than one delivery, e.g., a therapeutically effective amount of an API to be delivered to a patient orally may be included in a single tablet or capsule or multiple tablets or capsules and a therapeutically effective amount of an antigen to be provided by subcutaneous injection of a vaccine may be provided in a single injection or more than one injection. In some embodiments, the therapeutically effective amount could refer to an amount that is expected to provide the desired therapeutic effect. In some embodiments, the therapeutically effective amount refers to an amount provided to a patient determined to be effective during a clinical trial. In some embodiments, the therapeutically effective amount could be determined via clinical trials, pK modeling, or an FDA approved dose.

T_{end}: As used herein, the term “T_{end}” refers to the time at which plasma API concentration is greater than or equal to 0.5*Rₘₐₓ.

Tₛₜₐᵣₜ: As used herein, the term “Tₛₜₐᵣₜ” refers to the time at which plasma API concentration is greater than or equal to 0.9*Rₘₐₓ.

Vaccine: As used herein, the term “vaccine” or “vaccine composition” refers to a composition used to stimulate the production of antibodies and provide immunity against one or several diseases, prepared from the causative agent of a disease, its products, or a synthetic substitute, treated to act as an antigen without inducing the disease. A vaccine composition may include at least one antigen in a pharmaceutically acceptable vehicle useful for inducing an immune response in a subject. The vaccine composition is administered by doses and techniques known to those skilled in the pharmaceutical or veterinary fields, taking into account factors such as the age, sex, weight, species, and condition of the recipient animal and the route of administration.

The present invention provides methods of treatment that boost the immune responses of mRNA vaccines by providing sustained delivery of the vaccine antigen. Sustained delivery of mRNA vaccines aims to more closely mimic antigen presentation kinetics observed with an acute natural infection, as these infections induce strong and durable humoral immune responses. During acute infections, pathogenic antigens are present for several days to weeks resulting in the stimulation of the innate immune system and activation of the adaptive immune system. Subunit protein vaccines utilizing sustained delivery have focused on the use of repeated injections, osmotic pumps, or prototype microneedle patches to investigate the impact of antigen kinetics on immune responses. Results from these studies suggest that prolonged antigen availability leads to increased antigen retention in lymph nodes as well as increased Tfh cell and germinal center B cells. (See, e.g., Boopathy, A. V. et al. Enhancing humoral immunity via sustained-release implantable microneedle patch vaccination. Proceedings of the National Academy of Sciences of the United States of America 116, 16473-16478, (2019); Joyce, J. C. et al. Extended delivery of vaccines to the skin improves immune responses. J. Controlled Release 304, 135-145, (2019); DeMuth, P. C., Min, Y., Irvine, D. J. & Hammond, P. T. Implantable silk composite microneedles for programmable vaccine release kinetics and enhanced immunogenicity in transcutaneous immunization. Adv. Healthc. Mater. 3, 47-58, (2014); Tam, H. H. et al. Sustained antigen availability during germinal center initiation enhances antibody responses to vaccination. Proceedings of the National Academy of Sciences of the United States of America 113, (2016)).

However, unlike subunit protein vaccines where the antigen is immediately available for immune cell detection and rapidly cleared after injection, mRNA vaccines require intracellular processing from the host to produce the antigen of interest. Accordingly, sustained delivery (i.e., split-dose administration) of an mRNA vaccine may better recapitulate the kinetics of a natural viral infection and therefore improve the humoral immune response of mRNA vaccines.

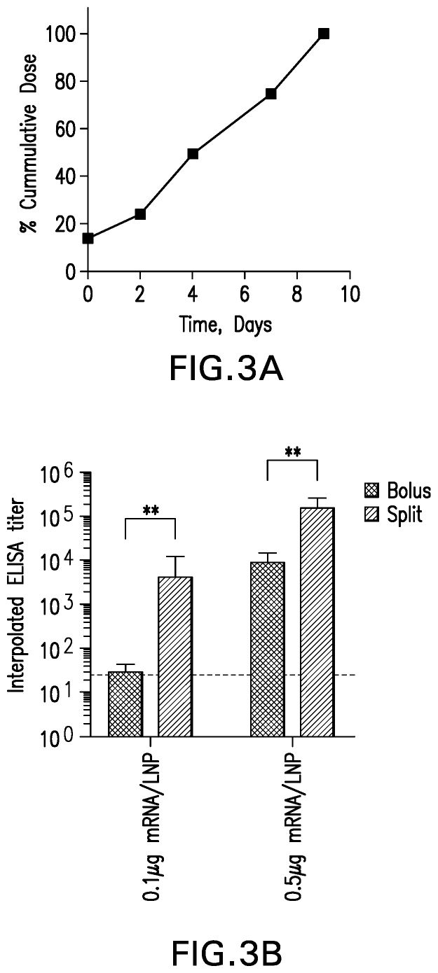

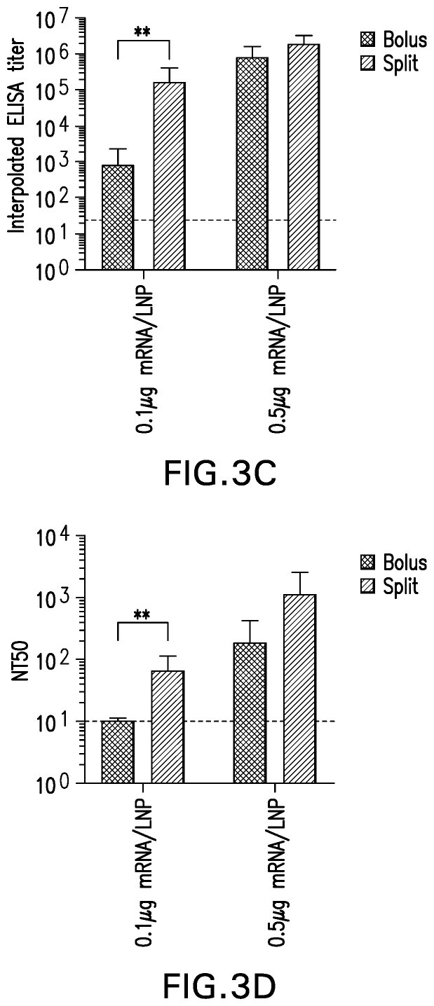

In some embodiments of the invention, mRNA/LNP vaccines are used to achieve improved humoral immunity by modulating the kinetics of mRNA-translated antigen expression and antigen presentation. For example, a RSV pre-F mRNA/LNP vaccine was used to compare split-vs. bolus-dose intradermal administration regimens (see Example 4). It was found that extended administration of an mRNA/LNP vaccine over a 9-day period in mice, using various split-dose schedules, resulted in significant enhancement of antibody titers (FIG. 3B-C, 6A-B) relative to bolus administration. Surprisingly, it was found that a low mRNA dose (0.1 μg) in split-dose vaccine groups resulted in greater than 10-fold and 100-fold improvements in ELISA titers when evaluated at days 21 and 42, respectively.

It was also surprisingly found that split-dose administration of an mRNA vaccine provided a dose sparing effect (See Example 6). In particular, a vaccine prepared according to the procedure outlined in Example 1 including a 0.1 μg dose of an API including mRNA administered as a split-dose elicited comparable antibody titers compared to a 0.5 μg dose of an API including mRNA administered as a bolus.

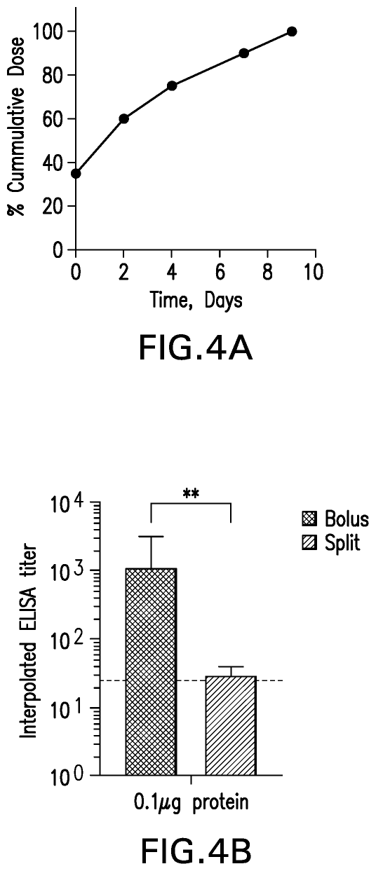

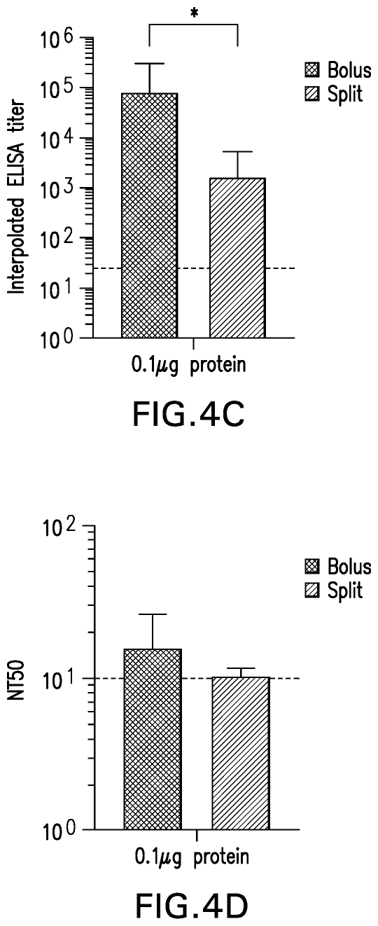

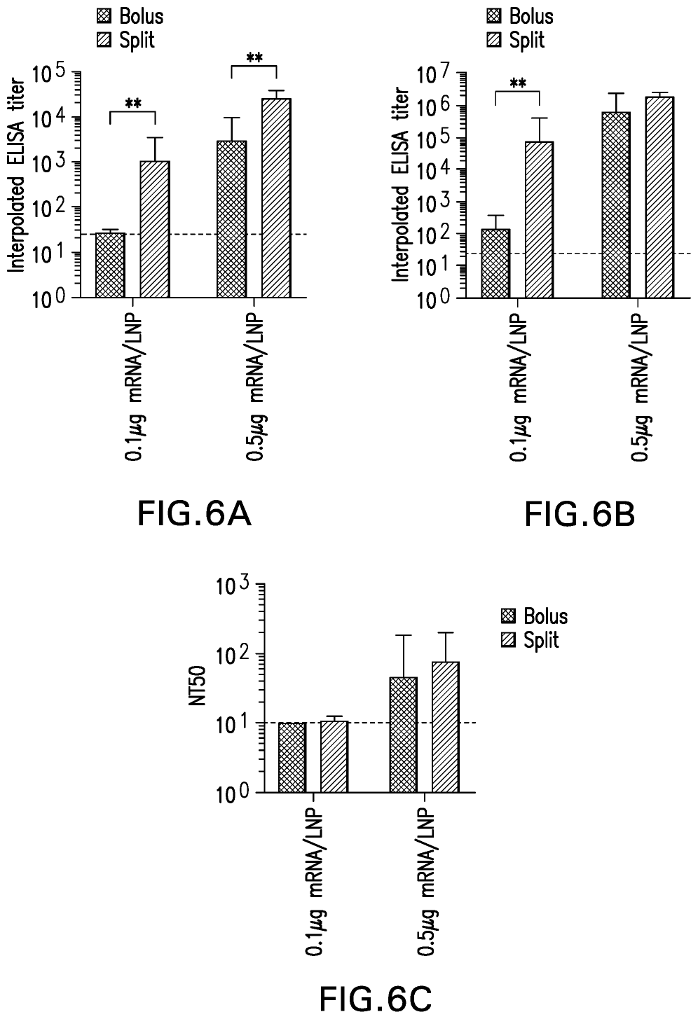

The first split-dose immunization regimen studied recapitulated a gradual increase in vaccine delivery of an mRNA vaccine comprising an mRNA encoding RSV pre-F protein, such as that provided in SEQ ID No. 2, with a low initial ‘burst’ release of the vaccine and sustained release rate being achieved by day 4 post application (see FIG. 3A and Example 4). The second immunization regimen mimicked a more significant initial ‘burst’, with 35% of the total dose being delivered immediately, followed by sustained delivery over remaining time course (see FIG. 4A and Example 4). Both split-dose regimens resulted in improved antibody titers compared to traditional bolus injections. While not statistically significant, the data suggest that the greater improvement over bolus administration is seen when the antigen delivery rate is more gradually increased over time with limited initial burst (see FIGS. 3A-3E and 6A-C, and examples 4-7).

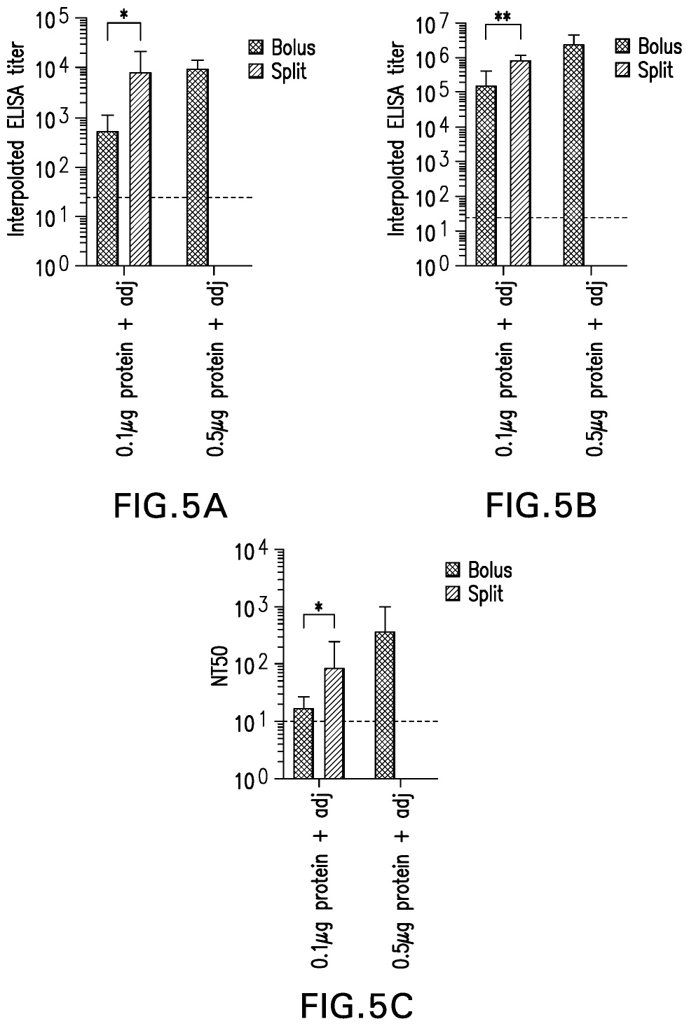

An RSV pre-F subunit protein, DS-Cav1, with and without adjuvant was analyzed to directly compare to the responses elicited by the RSV pre-F mRNA/LNP vaccine (see Example 7). While subunit protein vaccines may be preferred due to their improved safety profile over live-attenuated and inactivated viruses, they usually elicit inferior, shorter-lived immune response unless co-dosed with an adjuvant. One factor leading to their poor inherent immunogenicity is their rapid clearance following a single bolus injection. As seen in FIGS. 4A-4D and FIGS. 5A-5C, even with sustained vaccine delivery over a period of 9 days, the DS-Cav1 protein required co-formulation with an adjuvant to provide improved humoral immunity over traditional bolus administration of the same vaccine formulation (see FIGS. 4 and 5 and Example 6). Additionally, the split-dose RSV pre-F mRNA/LNP vaccine showed comparable antibody titers to the adjuvant-containing RSV subunit protein vaccine and a greater improvement in humoral immune response when compared to its respective bolus dose group (see FIGS. 5 and 6 and Example 6).

As shown herein, sustained delivery of an mRNA/LNP vaccine results in significant improvement in humoral immune responses and provides the opportunity to develop dose sparing mRNA vaccine regimens.

mRNA

As used herein, the term “messenger RNA” or “mRNA” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, mRNA used in the present invention may be purified to remove undesirable impurities including various enzymes and other reagents used during mRNA synthesis. The present invention may use mRNAs of a variety of lengths. In some embodiments, the present invention mRNA of or greater than about 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length may be included. In some embodiments, the present invention mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length may be included.

In some embodiments, mRNAs used in the present invention may include a 5′ cap structure. In some embodiments, the 5′ cap is added by an RNA terminal phosphatase removing one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. 2′-O-methylation may also occur at the first base and/or second base following the 7-methyl guanosine triphosphate residues. Examples of cap structures include, but are not limited to, m7GpppNp-RNA, m7GpppNmp-RNA and m7GpppNmpNmp-RNA (where m indicates 2′-Omethyl residues).

In some embodiments, the mRNA is an antigen-providing mRNA. In one embodiment, the mRNA has at least one open reading frame that can be translated by a cell or an organism provided with that mRNA. The product of this translation is a peptide or protein that may act as an antigen.

In one embodiment, the mRNA includes SEAP, such as that provided by SEQ ID NO.: 1. In one embodiment, the mRNA includes pre-F protein, such as that provided by SEQ ID NO.: 2.

### LNP

Lipid nanoparticles (LNPs) useful in the methods of the present invention are used herein to boost the immunological response of a vaccine, e.g. an mRNA vaccine. Generally, LNPs used in the present invention include one or more cationic lipids, one or more polymer-lipid conjugates (e.g., a poly(ethyleneglycol)-lipid (PEG-lipid)), one or more cholesterol, and one or more phospholipid.

In some embodiments, the LNP includes any cationic lipid mentioned in U.S. Patent Application Publication Nos. US 2008/0085870, US 2008/0057080, US 2009/0263407, US 2009/0285881, US 2010/0055168, US 2010/0055169, US 2010/0063135, US 2010/0076055, US 2010/0099738, US 2010/0104629, US 2013/0017239, and US 2016/0361411, International Patent Application Publication Nos. WO2011/022460 A1; WO2012/040184, WO2011/076807, WO2010/021865, WO2009/132131, WO2010/042877, WO2010/146740, and WO2010/105209, and in U.S. Pat. Nos. 5,208,036, 5,264,618, 5,279,833, 5,283,185, 6,890,557, and 9,669,097.

In some embodiments, the LNP includes a cationic lipid having the following structure, illustrated by Formula I:

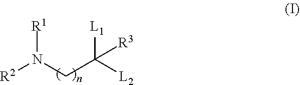

wherein:
R¹ and R² are each methyl;
R³ is H;
n is 1 or 2;
L₁ is selected from C₈-C₂₄ alkyl and C₈-C₂₄ alkenyl; and
L₂ is selected from C₄-C₉ alkyl and C₄-C₉ alkenyl;
or any pharmaceutically acceptable salt or stereoisomer thereof.

In some embodiments, the cationic lipid is an aminoalkyl lipid. In some embodiments, the cationic lipid is an asymmetric aminoalkyl lipid. In some embodiments, the cationic lipid is (13Z, 16Z)-N, N-dimethyl-3-nonyldocosa 13, 16-dien-1-amine (See, U.S. Pat. No. 9,669,097).

In some embodiments, the LNP includes 30-65 mole % cationic lipid. In some embodiments, the LNP includes 30-55 mole % cationic lipid. In some embodiments, the LNP includes 30-45 mole % cationic lipid. In some embodiments, the LNP includes 55-65 mole % cationic lipid. In some embodiments, the LNP includes 58 mole % cationic lipid.

In some embodiments, the LNP includes a neutral lipid selected from: phospholipids, diaeylphosphatidylcholine, diacylphosphatidyletbanolamine, ceramide, sphingomyelin, cephalin, cholesterol, cerebrosides, diacylglycerols, and combinations thereof. In some embodiments, the neutral lipid includes a phospholipid and cholesterol.

In some embodiments, the neutral lipid includes a sterol, such as cholesterol. In some embodiments, the neutral lipid includes cholesterol. In some embodiments, the LNP includes 10-40 mole % cholesterol. In some embodiments, the LNP includes 15-25 mole % cholesterol. In some embodiments, the LNP includes 10-20 mole % cholesterol. In some embodiments, the LNP includes 20-30 mole % cholesterol. In some embodiments, the LNP includes 10-15 mole % cholesterol. In some embodiments, the LNP includes 25-35 mole % cholesterol. In some embodiments, the LNP includes 30 mole % cholesterol.

In some embodiments, the LNP includes a phospholipid selected from:
phospholipids, aminolipids and sphingolipids. In some embodiments, the LNP includes a phospholipid selected from: phosphatidylcholine, phosphatidylethanolamine, phosphatidylserine, phosphatidylinositol, phosphatidic acid, palmitoyloleryl phosphatidylcholine, lysophosphatidylcholine, lysophosphatidylethanolamine, dipalmitoylphosphatidylcholine, dioleoylphosphatidylcholine, dstearoylphosphatidylcholine and dilinoleoylphosphatidylcholine. In some embodiments, the LNP includes a neutral lipid selected from: sphingolipid, glycosphingolipid families, diacylglycerols and S-acyloxyacids. In some embodiments, the LNP includes a neutral lipid selected from: phosphatidylcholine (PC), phosphatidylethanolamine (PE), and phosphatidylglycerol (PG), phosphatidylserine (PS), phosphatidylinositol (PI), phosphatidic acid (phosphatidate) (PA), dipalmitoylphosphatidylcholine, monoacyl-phosphatidylcholine (lyso PC), 1-palmitoyl-2-oleoyl-sn-glycero-3-phosphocholine (POPC), N-acyl-PE, phosphoinositides, and phosphosphingolipids. In some embodiments, the LNP includes a neutral lipid selected from: phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), and phosphatidylserine (DOPS). In some embodiments, the LNP includes a neutral lipid selected from: fatty acids include C14:0, palmitic acid (C16:0), stearic acid (C18:0), oleic acid (C18:1), linoleic acid (C18:2), linolenic acid (C18:3), arachidonic acid (C20:4), C20:0, C22:0 and lecithin. In some embodiments, the phospholipid includes 1,2-Distearoyl-sn-glycero-3-phosphocholine (DSPC). In some embodiments, the phospholipid includes a diether DSPC (e.g. (2R)-2,3-Bis(octadecyloxy) propyl 2-(trimethylazaniumyl)ethyl phosphate) In some embodiments of the methods of the invention, the phospholipid is represented by the structure set forth in Formula II:

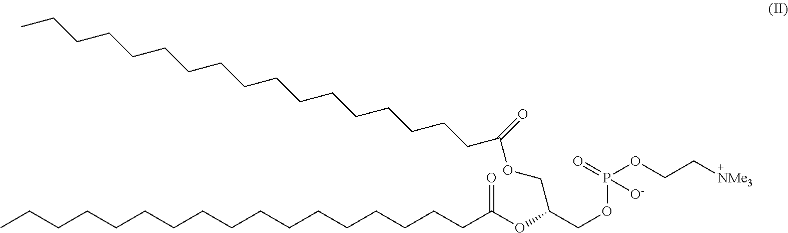

In some embodiments of the methods of the invention, the phospholipid is represented by the structure set forth in Formula III:

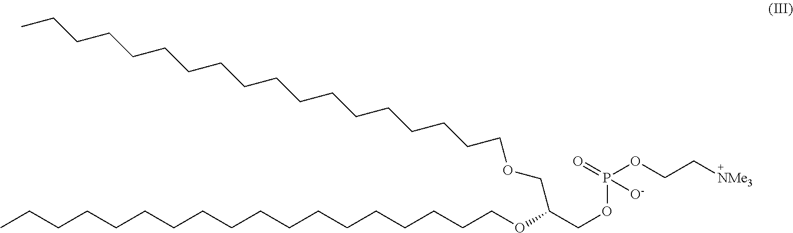

In some embodiments of the methods of the invention, the phospholipid is represented by the structure set forth in Formula IV:

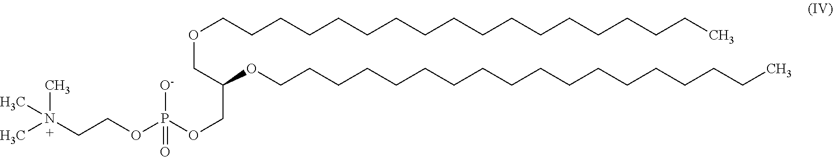

In some embodiments, the LNP includes 5-30 mole % phospholipid. In some embodiments, the LNP includes 5-15 mole % phospholipid. In some embodiments, the LNP includes 10-20 mole % phospholipid. In some embodiments, the LNP includes 20-30 mole % phospholipid. In some embodiments, the LNP includes 10-15 mole % phospholipid. In some embodiments, the LNP includes 25-30 mole % phospholipid. In some embodiments, the LNP includes 10 mole % phospholipid.

In some embodiments of the methods of the invention, the LNP includes a PEG-lipid. In some embodiments the PEG is conjugated to the lipid via a direct linkage (see, e.g., cPEG2000-DMG described below) or is conjugated to the lipid via linker (see, e.g., ePEG2000-DMG). In some embodiments, the PEG-lipid is conjugated to a diacylglycerol (a PEG-DAG). In some embodiments, the PEG is conjugated to DAG as described in, e.g., U.S. Patent Publication Nos. 2003/0077829 and 2005/008689. In one embodiment, the PEG-DAG conjugate is a PEG dimyristylglycerol (c14) conjugate. In some embodiments, the PEG-lipid is PEG-dimyristolglycerol (PEG-DMG). In certain embodiments of the methods of the invention, the PEG-lipid is PEG conjugated to dimyristoylglycerol (PEG-DMG), e.g., as described in Abrams et al., 2010, Molecular Therapy 18 (1): 171, and U.S. Patent Application Publication Nos. US 2006/0240554 and US 2008/0020058.

In certain embodiments of the methods of the invention, the PEG-lipid comprises a polyethylene glycol having an average molecular weight raining of about 500 daltons to about 10,000 daltons, of about 75 daltons to about 5,000 daltons, of about 1,000 daltons to about 5,000 daltons, of about 1,500 daltons to about 3,000 daltons or of about 2,000 daltons. In certain embodiments, the PEG-lipid comprises PEG1500, PEG2000 or PEG5000.

In some embodiments, the LNP includes 0.05-5 mole % polymer-lipid conjugate. In some embodiments, the LNP includes 1˜4 mole % polymer-lipid conjugate. In some embodiments, the LNP includes 0.5-2 mole % polymer-lipid conjugate. In some embodiments, the LNP includes 1˜4 mole % polymer-lipid conjugate. In some embodiments, the LNP includes 1-3 mole % polymer-lipid conjugate. In some embodiments, the LNP includes 1-2.5 mole % polymer-lipid conjugate. In some embodiments, the LNP includes 2 mole % polymer-lipid conjugate. In each case, it is expressed as total mole % of lipid in the particle.

In some embodiments, the LNP includes 30-65 mole % cationic lipid, 10-30 mole % cholesterol, 5-30 mole % phospholipid, and 0.05-4 mole % PEG-lipid. In some embodiments, the LNP includes 55-65 mole % cationic lipid, 25-35 mole % cholesterol, 5-15 mole % phospholipid, and 1-2.5 mole % PEG-lipid. In some embodiments, the LNP includes 40-50 mole % cationic lipid, 15-20 mole % cholesterol, 18-20 mole % phospholipid, and 1.5-2.5 mole % PEG-lipid. In some embodiments, the LNP includes 56-59 mole % cationic lipid, 15-20 mole % cholesterol, 18-20 mole % phospholipid, and 0.5-1.5 mole % PEG-lipid. In some embodiments, the LNP includes 56-59 mole % cationic lipid, 28-32 mole % cholesterol, 8-12 mole % phospholipid, and 1-3 mole % PEG-lipid. In some embodiments, the LNP includes 58 mole % cationic lipid, 30 mole % cholesterol, 10 mole % PEG-lipid and 2 mole % PEG-lipid.

In some embodiments of the methods of the invention, the LNP includes a PEG-lipid represented by the structure set forth in Formula V:

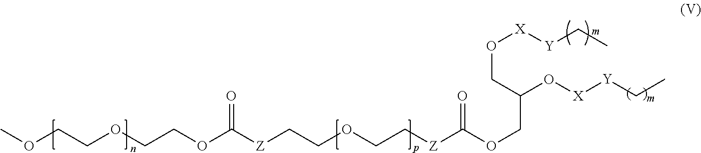

wherein:
each m is independently from 5-20;
n is from 20-60;
p is 0, 1, or 2;
each X is independently CH₂, CHR, CR₂, or C═O;
each Y is independently CH₂, CHR, CR₂, or NH;
each Z is independently absent, CH₂, or NH; and
each R is independently alkyl, aryl, heteroalkyl, or heteroaryl.

In some embodiments of the methods of the invention, the LNP includes a PEG-lipid represented by the structure set forth in Formula V, wherein each m is independently from 8-18. In some embodiments of the methods of the invention, the PEG-lipid is represented by the structure set forth in Formula V, wherein each m is independently from 10-15. In some embodiments of the methods of the invention, the PEG-lipid is represented by the structure set forth in Formula V, wherein each m is independently from 12-15. In some embodiments of the methods of the invention, the PEG-lipid is represented by the structure set forth in Formula V, wherein each m is independently 10, 11, 12, 13, 14, 15, 16, 17, 18, 19, or 20.

In some embodiments, the LNP includes a PEG-lipid represented by the structure set forth in Formula V, wherein n is from 20-60. In some embodiments of the methods of the invention, the PEG-lipid is represented by the structure set forth in Formula V, wherein n is from 20-50. In some embodiments of the methods of the invention, the PEG-lipid is represented by the structure set forth in Formula V, wherein n is from 20-45. In some embodiments of the methods of the invention, the PEG-lipid is represented by the structure set forth in Formula V, wherein n is from 30-60. In some embodiments of the methods of the invention, the PEG-lipid is represented by the structure set forth in Formula V, wherein n is from 30-50. In some embodiments of the methods of the invention, the PEG-lipid is represented by the structure set forth in Formula V, wherein n is from 30-45. In some embodiments of the methods of the invention, the PEG-lipid is represented by the structure set forth in Formula V, wherein n is from 35-60. In some embodiments of the invention, the PEG-lipid is represented by the structure set forth in Formula V, wherein n is from 35-50. In some embodiments of the invention, the PEG-lipid is represented by the structure set forth in Formula V, wherein n is from 35-45. In some embodiments of the invention, the PEG-lipid is represented by the structure set forth in Formula V, wherein n is from 40-60. In some embodiments of the methods of the invention, the PEG-lipid is represented by the structure set forth in Formula V, wherein n is from 40-55. In some embodiments of the methods of the invention, the PEG-lipid is represented by the structure set forth in Formula V, wherein n is from 40-50. In some embodiments, of the invention the PEG-lipid is represented by the structure set forth in Formula V, wherein n is from 40-55.

In some embodiments, the LNP includes a PEG-lipid represented by the structure set forth in Formula V, wherein p is 0, 1, or 2. In some embodiments of the invention, the PEG-lipid is represented by the structure set forth in Formula V, wherein p is 0. In some embodiments of the invention, the PEG-lipid is represented by the structure set forth in Formula V, wherein p is 1. In some embodiments of the invention, the PEG-lipid is represented by the structure set forth in Formula V, wherein p is 2.

In some embodiments, the LNP includes a PEG-lipid represented by the structure set forth in Formula V, wherein each X is independently CH₂, CHR, CR₂, or C═O and R is alkyl, aryl, heteroalkyl, or heteroaryl. In some embodiments of the invention, the PEG-lipid is represented by the structure set forth in Formula V, wherein each X is independently CH₂. In some embodiments of the invention, the PEG-lipid is represented by the structure set forth in Formula V, wherein each X is independently CHR and wherein R is alkyl, aryl, heteroalkyl, or heteroaryl. In some embodiments of the invention, the PEG-lipid is represented by the structure set forth in Formula V, wherein each X is independently CR₂ and R is alkyl, aryl, heteroalkyl, or heteroaryl.

In some embodiments, the LNP includes a PEG-lipid represented by the structure set forth in Formula V, wherein each Y is independently CH₂, CHR, CR₂, or NH and wherein R is alkyl, aryl, heteroalkyl, or heteroaryl. In some embodiments of the invention, the PEG-lipid is represented by the structure set forth in Formula V, wherein each Y is CH₂. In some embodiments of the invention, the PEG-lipid is represented by the structure set forth in Formula V, wherein each Y is independently CHR and wherein R is alkyl, aryl, heteroalkyl, or heteroaryl. In some embodiments of the invention, the PEG-lipid is represented by the structure set forth in Formula V, wherein each Y is independently CR₂ and R is alkyl, aryl, heteroalkyl, or heteroaryl. In some embodiments of the invention, the PEG-lipid is represented by the structure set forth in Formula V, wherein each Y is NH.

In some embodiments, the LNP includes a PEG-lipid represented by the structure set forth in Formula V, wherein each Z is independently absent, CH₂, or NH. In some embodiments of the invention, the PEG-lipid is represented by the structure set forth in Formula V, wherein each Z is absent. In some embodiments of the invention, the PEG-lipid is represented by the structure set forth in Formula V, wherein each Z is CH₂. In some embodiments of the invention, the PEG-lipid is represented by the structure set forth in Formula V, wherein each Z is independently NH.

In some embodiments, the LNP includes a PEG-lipid represented by the structure set forth in Formula V, wherein R is alkyl, aryl, heteroalkyl, or heteroaryl.

In some embodiments, the LNP includes a PEG-lipid represented by the structure set forth as Formula VI:

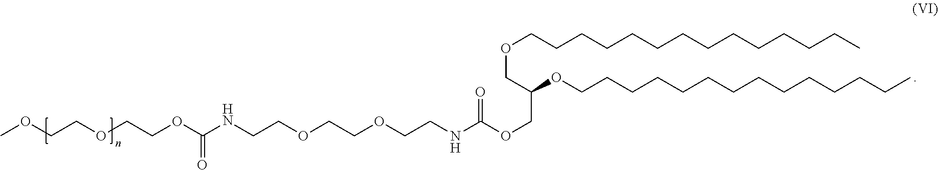

In some embodiments, the LNP includes a PEG-lipid represented by the structure set forth as Formula VII:

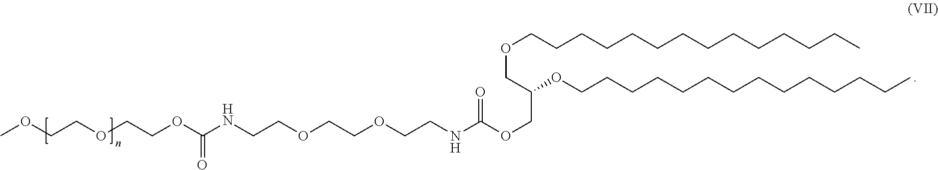

In some embodiments, the LNP includes a PEG-lipid represented by the structure set forth as Formula VIII:

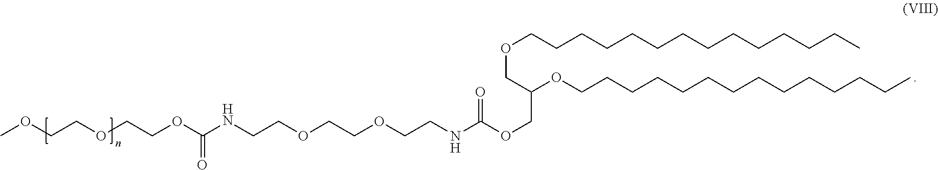

In some embodiments, the LNP includes a PEG-lipid represented by the structure set forth as Formula IX:

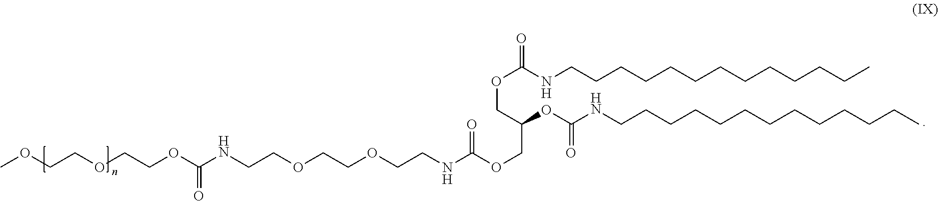

In some embodiments, the LNP includes a PEG-lipid represented by the structure set forth as Formula X:

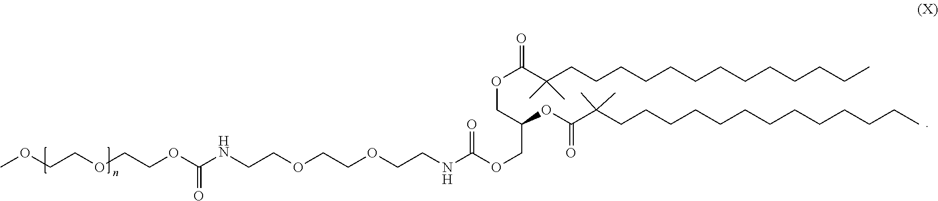

In some embodiments, the LNP includes a PEG-lipid represented by the structure set forth as Formula XI:

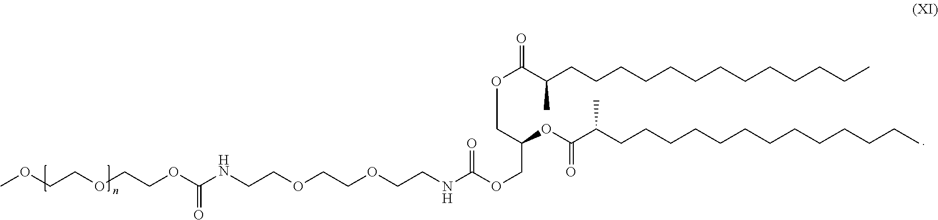

In some embodiments, the LNP includes a PEG-lipid represented by the structure set forth as Formula XII:

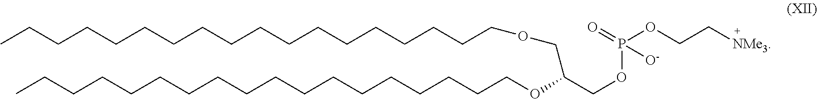

In some embodiments, a composition of the present invention includes an LNP that includes a buffer. In some embodiments, the buffer is selected from any pharmaceutically acceptable buffer, including acetic acid, histidine, citrate, Bis-Tris, HEPES, phosphate, MES, and combinations thereof. In some embodiments, the buffer is present in the amount of 1 mMol (mM) to about 100 mMol.

In some embodiments, a composition of the present invention includes an LNP that includes a tonicity modifier. In some embodiments, the tonicity modifier is selected from any pharmaceutically acceptable tonicity modifiers, such as sodium chloride, potassium chloride, sucrose, trehalose and combinations thereof. In some embodiments, the tonicity modifier is present in an amount of 10 mM to 500 mM.

In some embodiments, a composition of the present invention includes an LNP that includes a cryoprotectant. In some embodiments, the cryoprotectant is selected from any pharmaceutically acceptable cryoprotectants, such as sucrose, trehalose, mannitol, glycerol, and the like, and combinations thereof. In some embodiments, the cryoprotectant is present in the amount of 0.1 to about 10% (w/v).

### Methods of Making LNP

In some embodiments, the methods of the invention include the use of an LNP, which may increase the immune response to the antigen of interest or decrease the amount of API required to bring about the desired therapeutic effect, e.g. immune response. The LNP may be formed, for example, by a rapid precipitation process that entails micro-mixing the lipid components dissolved in a lower alkanol solution (e.g. ethanol) with an aqueous solution using a confined volume mixing apparatus such as a confined volume T-mixer, a multi-inlet vortex mixer, microfluidics mixer devices, or other. The lipid solution may include one or more cationic lipids, one or more neutral lipid (e.g., phospholipids, DSPC, cholesterol), one or more polymer-lipid conjugate (e.g. cPEG2000-DMG, cPEG-2000-DMG(s), ePEG2000-DMG, ether-ePEG2000-DMG) at specific molar ratios in ethanol.

In some embodiments, the aqueous and organic solutions are optionally heated to a temperature in the range of 25° C.-45° C., preferably 30° C.-40° C., and then mixed in a confined volume mixer to form the LNP. When a confined volume T-mixer is used, the T-mixer may have an internal diameter range from 0.25 to 10.0 mm. In some embodiments, the alcohol and aqueous solutions are delivered to the inlet of the T-mixer using programmable syringe pumps, and with a total flow rate from 10 mL/min-600 L/minute. In some embodiments, the aqueous and alcohol solutions are combined in the confined-volume mixer with a ratio in the range of 1:1 to 4:1 vol: vol. In some embodiments, the aqueous and alcohol solutions are combined at a ratio in the range of 1.1:1 to 4:1, 1.2:1 to 4:1, 1.25:1 to 4:1, 1.3:1 to 4:1, 1.5:1 to 4:1, 1.6:1 to 4:1, 1.7:1 to 4:1, 1.8:1 to 4:1, 1.9:1 to 4:1, 2.0:1 to 4:1, 2.5:1 to 4:1, 3.0:1 to 4:1, and 3.5:1 to 4:1.

In some embodiments, the combination of ethanol volume fraction, solution flow rates, lipid(s) concentrations, mixer configuration and internal diameter, and mixer tubing internal diameter utilized at this mixing stage provide LNPs having a particle size of the between 30 and 300 nm. The resulting LNP suspension may be diluted into higher pH buffers in the range of 6-8. In some embodiments, the diluted suspension is further diluted with an additional buffer, such as phosphate buffered saline having a pH between 6-8.

In some embodiments, the LNPs are concentrated and filtered via an ultrafiltration process to remove the alcohol. In some embodiments, the high pH buffer is removed and exchanged for a final buffer solution. In some embodiments, the final buffer solution isa phosphate buffered saline or any buffer system suitable for cryopreservation (for example, buffers containing sucrose, trehalose or combinations thereof). Following filtration, the vialed LNP product may be stored under suitable storage conditions (such as, 2° C.-8° C., or −80 to −20° C. if frozen) or may be lyophilized.

In some embodiments, the ultrafiltration process includes a tangential flow filtration format (“TFF”) that utilizes a hollow fiber membrane nominal molecular weight cutoff range from 30-500 KD, targeting 500 KD. In some embodiments, the TFF retains the LNP in the retentate and the filtrate or permeate contained the alcohol and final buffer wastes. Following initial concentration, the LNP may be diafiltered against the final buffer (for example, phosphate buffered saline “PBS”) to remove the alcohol and perform buffer exchange. The material may then be concentrated via ultrafiltration to a final desired concentration.

In some embodiments, the concentrated LNP is then filtered to reduce bioburden into a suitable container under aseptic conditions. In some embodiments, the bioburden reduced filtration (BRF) is accomplished by passing the LNP suspension through a pre-filter (Sartobran P 0.45 uin capsule) and a bioburden reduction filter (Sartobran P 0.2 win capsule). Following filtration, the LNP bulk intermediate may be stored under suitable conditions.

### Methods of Treatment of the Invention

Also provided herein is a method of treating a disease or disorder in a patient in need thereof including providing an active pharmaceutical ingredient (API) to said patient comprising: (a) administering a first split-dose of said API; (b) waiting for a pre-determined amount of time to pass; (c) administering an additional split-dose of said API; and optionally repeating steps (b) and (c); wherein each split-dose comprises an amount of API that is less than the amount of the API that is determined to be effective at treating the disease or disorder via a bolus dose.

In some embodiments, a method of inducing an immune response in a patient is provided including providing an active pharmaceutical ingredient (API) to said patient comprising: (a) administering a first split-dose of said API; (b) waiting for a pre-determined amount of time to pass; (c) administering an additional split-dose of said API; and optionally repeating steps (b) and (c), wherein each split-dose comprises an amount of API that is less than the amount of API that is determined to be effective at inducing an immune response via a bolus dose.

In some embodiments, a method of inducing a protective immune response in a patient is provided including providing an active pharmaceutical ingredient (API) to said patient comprising (a) administering a first split-dose of said API; (b) waiting for a pre-determined amount of time to pass; (c) administering an additional split-dose of said API; and optionally repeating steps (b) and (c); wherein each split-dose comprises an amount of API that is effective at inducing a protective immune response that is less than the amount of API that is determined to be effective at inducing an immune response via a bolus dose. In some embodiments, the protective immune response includes preventing infection, preventing disease, decreasing the likelihood of infection or disease, and/or decreasing the amount or severity of symptoms/clinical manifestations of the disease.

In some embodiments, the amount of API (e.g. mRNA) delivered to the patient in each administration of the split-dose is less than 100% of the amount of API delivered to the patient in a bolus dose. In some embodiments, the amount of API delivered to the patient in each administration of the split-dose is equal 90% or less of the bolus dose. In some embodiments, the amount of API delivered to the patient in each administration of the split-dose is equal to 80% or less of the bolus dose. In some embodiments, the amount of API delivered to the patient in each administration of the split-dose is 70% or less of the bolus dose. In some embodiments, the amount of API delivered to the patient in each administration of the split-dose is equal to 60% or less of the bolus dose. In some embodiments, the amount of API delivered to the patient in each administration of the split-dose is equal to 50% or less of the bolus dose. In some embodiments, the amount of API delivered to the patient in each administration of the split-dose is equal to 40% or less of the bolus dose. In some embodiments, the amount of API delivered to the patient in each administration of the split-dose is equal to 30% or less of the bolus dose. In some embodiments, the amount of API delivered to the patient in each administration of the split-dose is equal to 20% or less of the bolus dose. In some embodiments, the amount of API delivered to the patient in each administration of the split-dose is equal to 10% or less of the bolus dose. In some embodiments, the amount of API delivered to the patient in each administration of the split-dose is equal to or less than 9%, 8%, 7%, 6%, 5%, 4%, 3%, 2%, or 1% of the bolus dose.

The amount of API in each split-dose can be the same or different than other split-doses in the same treatment regimen. In some embodiments, the pre-determined amount of time between each split dose is 1 day, 2 days, 3 days, 4 days, 5 days, one week, two weeks, three weeks, four weeks, one month, two months, three months, four months, 5 months, 6 months, 7 months, 8 months, 9 months, 10 months, 11, months, one year, two years, three years, etc. The amount of time between each split-dose can be the same or different. In some embodiments, the amount of API in a split dose is relative to a bolus dose. In some embodiments, the bolus dose can be an amount that was approved by the FDA or other regulatory agency as a dose expected to bring about a desired therapeutic effect. In some embodiments, the bolus dose could be determined through clinical trial(s). In some embodiments, the bolus dose could be determined through PK model-based simulations. In some embodiments, the bolus dose could be an amount expected or hypothesized to bring about a therapeutic effect as a bolus dose.

In embodiment 1, a method of treating a disease or disorder in a patient in need thereof is provided comprising: providing an active pharmaceutical ingredient (API) to said patient comprising: (a) administering a first split-dose of said API; (b) waiting for a pre-determined amount of time to pass; (c) administering an additional split-dose of said API; and optionally repeating steps (b) and (c); wherein each split-dose comprises an amount of API that is less than the amount of said API that is determined to be effective at treating said disease or disorder via a bolus dose.

In embodiment 2, the method of embodiment 1 is provided to the patient as an mRNA composition comprising an mRNA encoding an antigen and a pharmaceutically acceptable carrier.

In embodiment 3, the method of embodiment 2 is provided wherein the mRNA composition further comprises a lipid nanoparticle (LNP).

In embodiment 4, the method of embodiment 3 wherein the LNP comprises a cationic lipid, a phospholipid, cholesterol, and a PEG-lipid.

In embodiment 5, the method of any of embodiments 3-4 is provided wherein the LNP comprises 30-65 mole % cationic lipid, 5-30 mole % phospholipid, 10-40 mole % cholesterol, and 0.5-4 mole % PEG-lipid.

In embodiment 6, the method of any of embodiments 3-5 is provided wherein the LNP comprises 55-65 mole % cationic lipid, 5-15 mole % phospholipid, 25-35 mole % cholesterol, and 1-2.5 mole % PEG-lipid.

In embodiment 7, the method of any of embodiments 3-6 is provided wherein the LNP comprises DSPC, cholesterol, ePEG2000-DMG, and (13Z, 16Z)-N, N-dimethyl-3-nonyldocosa 13, 16-dien-1-amine.

In embodiment 8, the method of any of embodiments 3-7 is provided wherein the LNP comprises 5-15 mole % DSPC, 25-35 mole % cholesterol, 1-2.5 mole % ePEG2000-DMG, and 55-65 mole % (13Z, 16Z)-N, N-dimethyl-3-nonyldocosa 13, 16-dien-1-amine.

In embodiment 9, the method of any of embodiments 1-8 is provided wherein the total amount of API provided to the patient by administration of all split-doses is equal to X % of the amount of the API provided in a bolus dose of said API, wherein X is less than or equal to 100.

In embodiment 10, the method of any of embodiments 1-9 is provided wherein the amount of API in each split-dose is the same.

In embodiment 11, the method of any of embodiments 1-9 is provided wherein the amount of API in each split-dose is not the same.

In embodiment 12, the method of any of claims 1-11 is provided wherein the therapeutic effect is the same or greater than such effect when said API is provided to the patient as a bolus dose.

In embodiment 13, the method of any of embodiments 1-11 is provided wherein the ΔT_{1/2} of the API provided as a split-dose is greater than the ΔT_{1/2} when the API is provided as a bolus dose.

In embodiment 14, the method of any of embodiments 1-11 is provided wherein the ΔT_{1/2} of the API provided as a split-dose is at least 2-10 times greater than the ΔT_{1/2} when the API is provided as a bolus dose.

In embodiment 15, the method of any of embodiments 1-11 is provided wherein the Rₘₐₓ of the API provided as a split-dose is less than the Rₘₐₓ when the API is provided as a bolus dose.

In embodiment 16, the method of any of embodiments 1-11 is provided wherein the Rₘₐₓ of the API provided as a split-dose is at least 50% less than the Rₘₐₓ when the API is provided as a bolus dose.

In embodiment 17, the method of any of embodiments 1-11 is provided 20 wherein the AUC of the API provided as a split-dose is approximately the same as the AUC when the API is provided as a bolus dose.

In embodiment 18, a method of inducing an immune response is provided including providing an active pharmaceutical ingredient (API) to said patient comprising: (a) administering a first split-dose of said API; (b) waiting for a pre-determined amount of time to pass; (c) administering an additional split-dose of said API; and optionally repeating steps (b) and (c), wherein each split-dose comprises an amount of API that is less than the amount of API that is determined to be effective at inducing an immune response via a bolus dose.

In embodiment 19, a method of inducing a protective immune response is provided including providing an active pharmaceutical ingredient (API) to said patient comprising (a) administering a first split-dose of said API; (b) waiting for a pre-determined amount of time to pass; (c) administering an additional split-dose of said API; and optionally repeating steps (b) and (c); wherein each split-dose comprises an amount of API that is effective at inducing a protective immune response that is less than the amount of API that is determined to be effective at inducing an immune response via a bolus dose. In some embodiments, the protective immune response includes preventing infection, preventing disease, decreasing the likelihood of infection or disease, and/or decreasing the amount or severity

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

### Example 1: Preparation of Vaccine Compositions

mRNA Vaccine 1

mRNA Vaccine 1 included an ARCA capped and 5MeC substituted SEAP (SEQ ID NO.: 1) purchased from TriLink Biotech and an LNP including a cationic lipid, 1,2-distearoyl-sn-glycero-3-phosphocholine (DSPC), cholesterol, and poly(ethylene glycol) 2000-dimyristoylglycerol (PEG2000-DMG) (See Espeseth, A. S. et al., NPJ Vaccines, 220 5, 16) and were prepared by rapid nanoprecipitation following the procedure described in Gindy, M. E. et al., Mol. Pharmaceutics 2014, 11 (11) 4143-53.

mRNA Vaccine 2

mRNA Vaccine 2 included CleanCap® RSV pre-F mRNA (SEQ ID NO.: 2) purchased from TriLink Biotech and an LNP including a cationic lipid, 1,2-distearoyl-sn-glycero-3-phosphocholine (DSPC), cholesterol, and poly(ethylene glycol) 2000-dimyristoylglycerol (PEG2000-DMG) (See Espeseth, A. S. et al., NPJ Vaccines, 220 5, 16) and were prepared by rapid nanoprecipitation following the procedure described in Gindy, M. E. et al., Mol. Pharmaceutics 2014, 11 (11) 4143-53. All mRNA vaccine formulations were tested for particle size, lipid concentration, mRNA concentration, and mRNA encapsulation prior to injection into animals.

### RSV Subunit Vaccine 1

An unadjuvanted RSV subunit pre-F protein vaccine, DS-Cav1, (hereinafter “RSV Subunit Vaccine 1”) was made similar to what has been previously described by McLellan J S, Chen M, Joyce M G, Sastry M, Stewart-Jones G B, Yang Y, et al., Structure-based design of a fusion glycoprotein vaccine for respiratory syncytial virus. Science 2013 Nov. 1; 342 (6158): 592-8. The DS-Cav1 and variant RSV F sequences were codon optimized for mammalian codon usage (Life Technologies), cloned into an expression vector, and transiently transfected into Expi293 suspension cells (Life Technologies). Cell culture supernatants were harvested day 3 to 7 post-plasmid transfection and evaluated in western blot and ELISA assays described below. To obtain purified RSV F proteins, cell culture supernatants were purified using a modified method based on the procedure previously described by Mclellan et al., Briefly, his-tagged proteins were purified using Ni-Sepharose chromatography (GE Healthcare). Tags were removed by overnight digestion with thrombin. Digestion was performed during dialysis to reduce imidazole concentration. To remove co-eluting contaminants and uncleaved F protein, samples were subjected to a second Ni-Sepharose chromatography step. F proteins were further purified by gel filtration chromatography (Superdex 200, GE Healthcare) and were stored in a buffer of 50 mM HEPES pH 7.5, 300 mM NaCl.

### RSV Subunit Vaccine 2

An adjuvanted RSV subunit pre-F protein vaccine including DS-Cav1 formulated with an LNP (hereinafter “RSV Subunit Vaccine 2”) was made by combining the RSV construct described in RSV Subunit Vaccine. 1 above and an LNP including a cationic lipid, 1,2-distearoyl-sn-glycero-3-phosphocholine (DSPC), cholesterol, and poly(ethylene glycol) 2000-dimyristoylglycerol (PEG2000-DMG) (See Espeseth, A. S. et al., NPJ Vaccines, 220 5, 16) and were prepared by rapid nanoprecipitation following the procedure described in Gindy, M. E. et al., Mol. Pharmaceutics 2014, 11 (11) 4143-53.

### Example 2: Population Pharmacokinetics/Pharmacodynamics (Pop PK/PD) Modeling

To determine the half-life of mRNA translation after bolus dose administration of mRNA Vaccine 1, the previously reported equation, t1/2=Δt×ln (2)/ln (NO/Nt), was utilized (Pardi, N. et al., J. Control Release, 2015, 217, 345-51). Equation parameters are defined as follows: Δt is the time between SEAP serum protein measurements, NO is the peak SEAP serum protein level and Nt is the SEAP serum protein level at measurement termination.

The population pharmacokinetic (“PK”)/pharmacodynamic (“PD”) analysis used to compare bolus and split-dose regimens was conducted in NONMEM using PsN 4.7.15 execution. A one-compartment model with a depot compartment and first-order absorption was used to describe the PK profiles of mRNA vaccine. A linear elimination was assumed. The PD model was linked to the central compartment concentration, and the response compartment was also regulated by the KIN and KOUT rate constants. Both additive and proportional residual errors were considered in the model. For modeling purposes, data points that were below the limit of quantification were assigned a value of half the lower limit of quantification. The performance of the model was evaluated using goodness-of-fit diagnostic plots and visual predictive check (200 simulations). R was applied for graphical diagnostics, simulations, response curve plots, and calculations of AUC, Rₘₐₓ, and ΔT_{1/2} under different dosing regimens. Under the simulation, ten groups of 10 individuals were subjected to a single bolus dose, a split-dose 1, and split-dose 2 scenario using an application of an RxODE R package. 1 μg and 10 μg dosing amounts were simulated separately. The sampling time was every 1 hour until 28 days after dose. AUC were calculated via an application of aPKNCA R package.

### Example 3: Serum Neutralization Assay

Serum neutralization assays were conducted using an AlphaLISA assay. Sera were heat inactivated for 30 minutes and serially diluted into a 1536-well plate. The sera were combined with RSV-A (long) to achieve a final concentration of 500 pfu/well. After 1 hour of incubation, HEp-2 cells were added to each well and further incubated for 72 h at 37° C. Cells were then lysed with AlphaLISA lysis buffer (Perkin Elmer) for 60 minutes at room temperature and then exposed to a suspension of AlphaLISA acceptor beads (Perkin Elmer) and biotinylated anti-RSV-F (b-FC) antibody (Perkin Elmer) prepared in 1× immunoassay buffer for 1 hour at room temperature. A suspension of streptavidin coated donor beads (Perkin Elmer) prepared in 1× immunoassay buffer was added to the plate and the plate was further incubated at room temperature for 30 minutes. Fluorescence was measured on a En Vision microplate reader (Perkin Elmer). Four-parameter curve fitting (GraphPad Prism® 7 software) was used to calculate titers.

### Example 4: Bolus Dosing of mRNA Vaccine Results in Rapid Protein Expression

Sustained delivery (i.e., split-dose) administration may better recapitulate the kinetics of a natural viral infection and therefore improve the humoral immune response of mRNA vaccines as has been previously demonstrated for subunit proteins. As discussed above, mRNA vaccines require intracellular processing from the host to produce the antigen of interest. This intracellular processing requirement could lead to delayed availability of the antigen and prolonged antigen exposure eliminating the benefit of sustained delivery as observed for subunit vaccines. To understand the kinetics of protein expression from mRNA vaccines following bolus administration, the expression of protein from mRNA Vaccine 1 following bolus administration in animals was characterized.

mRNA Expression Studies

Mouse studies were approved by the Institutional Animal Care and Use Committee at Merck & Co., Inc., Rahway, NJ, USA. BALB/c mice were obtained from Charles River Laboratories. Doses of mRNA Vaccine 1 were made as described above in Example 1. The kinetic mRNA expression study included BALB/c mice (10 mice/dose group), aged 8-9 weeks, that were immunized with mRNA Vaccine 1 via intramuscular injection in both quadricep muscles with 50 uL/quad. Blood draws were performed at indicated timepoints to conduct SEAP serum analysis.

SEAP activity was measured using Novabright Phospha-Light EXP Assay kit for SEAP Reporter Gene Detection (Invitrogen, N10578). Murine serum samples were heat inactivated at 56° C. for 30 minutes. Samples were then diluted 1:2 in PBS and 25 μl diluted serum was added in duplicate to a 96 well plate. A standard curve was generated using recombinant SEAP protein (InvivoGen, rec-hseap). Heat inactivated naïve mouse serum was diluted 1:2 in PBS and added to a separate 96 well plate to serve as a control. SEAP protein was added to the first well for a final concentration of 100 μg/well and then diluted 1:2 for a 15-point standard curve. 25 μl of each standard was added to the assay plate. 50 μl of a mixture of non-placental alkaline phosphatase inhibitors (“Component A”) was added to each well of the plate and the plate was incubated at 65° C. for 5 minutes. Next 50 μl of a composition including a CSPD® substrate and Emerald-III™ luminescence enhancer (“Component B”) was added to each well and the plate was incubated at room temperature for 17 minutes. Luminescence was read using a Versa Max plate reader. Serum SEAP concentrations for each sample was calculated using linear regression in GraphPad Prism® 7.

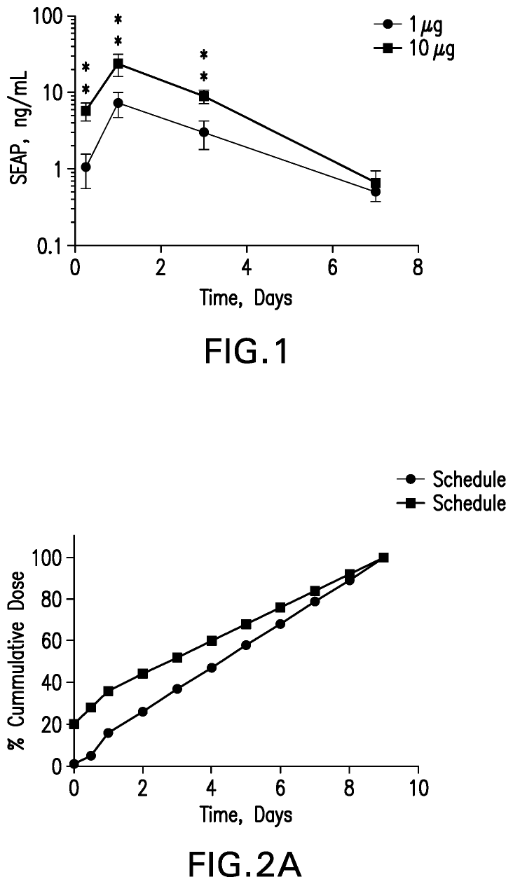

Bolus dosing of mRNA Vaccine 1 was achieved by immunizing BALB/c mice (10 mice, aged 8-9 weeks) at two dose levels (1 μg of 10 μg). The SEAP serum protein levels were measured over the course of 7 days using a chemiluminescent assay. The maximum serum concentration (Cmax) of SEAP protein for both high (10 μg) and low (1 μg) doses of vaccine were found to occur at approximately 24 hours and steadily decrease over time, with minimal protein detected at 7 days (FIG. 1). This indicated that protein expression following mRNA vaccination is initiated relatively rapidly. The half-life of mRNA-translated SEAP, calculated using the SEAP concentration versus time curve of FIG. 1, was estimated at 22.5 hours.

### Example 5: Computational Model of mRNA Vaccine-Induced Protein Expression Resulting from Sustained Delivery of mRNA

In this example, representations of vaccine sustained administration scenarios were mathematically modeled to understand the consequences of sustained administration or split-dose schedules on mRNA translated protein.

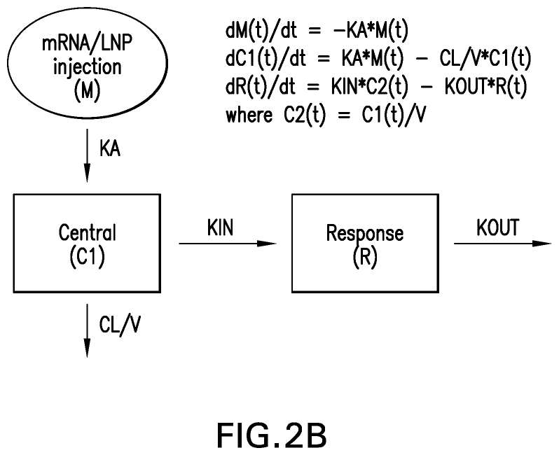

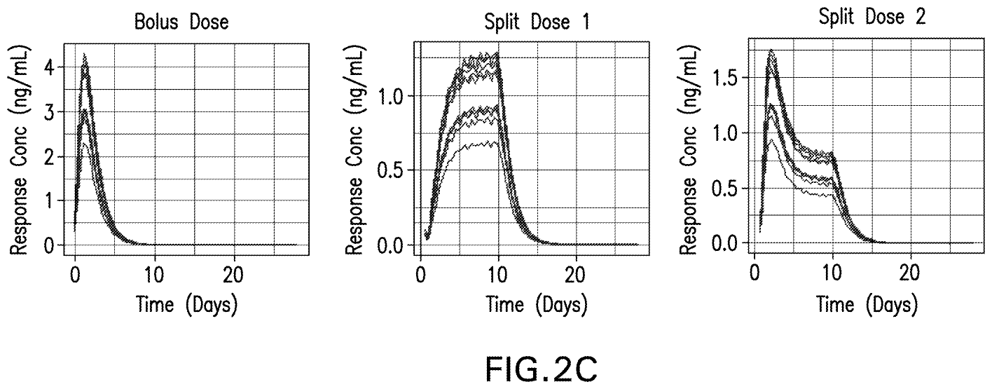

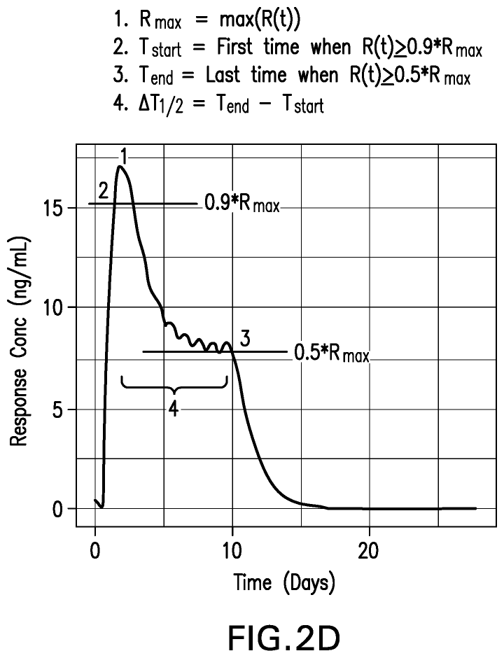

A population pharmacokinetic/pharmacodynamics (pop PK/PD) model was built to describe the SEAP protein expression kinetics induced by bolus administration of an mRNA vaccine and to simulate the response profiles under different split-dose regimens. The data shown in FIG. 1 and described in Example 4 was used to build the model. Two split-dose schedules were selected for study, each providing the same amount of API released over the time period indicated. See FIG. 2A. The first was a split-dose designed to mimic sustained release formulations that gradually release API without providing an initial burst of API (referred to hereinafter as “Schedule 1”). The second was a split-dose designed to mimic a release profile indicative of an initial burst of API followed by a gradual release API (referred to hereinafter as “Schedule 2”). Both Schedule 1 and Schedule 2 are examples of release profiles that may arise from long-acting injectable depots or microneedle patches to provided sustained administration and/or sustained administration coupled with solid-state stability of API, and in particular of APIs that include mRNA vaccines.

A basic one-compartment model with a depot compartment, first-order absorption and linear elimination was assumed for the PK profiles of mRNA (FIG. 2B). The response compartment, representing expressed SEAP protein concentration, was modulated by the central compartment concentration (C2) and the KIN and KOUT rate constants shown in the differential equations in FIG. 2B. The response curves representing expressed SEAP protein concentration for 100 simulated subjects under each of the three 1 μg dosing scenarios (single bolus dose, Schedule 1, and Schedule 2) are shown in FIG. 2C.

To quantify and summarize the effects of the different dosing regimens on protein expression, the area under the curve (AUC), the maximum response (Rmax), and the duration of elevated protein expression (defined as ΔT_{1/2} and illustrated in FIG. 2D using the response curve under split dose 2 scenario as an example) was calculated. Table I depicts the predicted ΔT_{1/2}, Rmax, and AUC values for bolus dose and split-dose schedules following 1 μg and 10 μg administration.

TABLE I

| | ΔT_{1/2} (days) | | Rₘₐₓ (ng/mL) | | AUC (day*ng/mL) | |
|---|---|---|---|---|---|---|
| Scenario | 10 μg | 1 μg | 10 μg | 1 μg | 10 μg | 1 μg |
| Bolus Dose | 1.9 | 1.9 | 37.7 ± 2.5 | 3.5 ± 0.2 | 101.4 ± | 9.5 ± 0.6 |
| | | | | | 6.7 | |
| Schedule 1 | 6.8 | 6.8 | 11.4 ± 0.8 | 1.1 ± 0.1 | 101.3 ± | 9.4 ± 0.5 |
| | | | | | 6.7 | |
| Schedule 2 | 5.3 | 4.4 | 15.5 ± 1.0 | 1.4 ± 0.1 | 101.1 ± | 9.2 ± 0.5 |
| | | | | | 6.6 | |

As shown in Table I, both Schedule 1 and Schedule 2 resulted in comparable AUCs to that seen after a single bolus dose administration. However, differences between bolus dose and each of the split-dose regimens were observed when evaluating Rmax and ΔT_{1/2}. The difference in Rmax was particularly statistically significant. A reduction in Rmax was observed across both 1 μg and 10 μg dosing concentrations following both Schedule 1 and Schedule 2 when compared to bolus dose administration. Schedule 1 and Schedule 2 also resulted in increased predicted durations of elevated SEAP protein expression (ΔT_{1/2}). Following 1 μg treatment, ΔT_{1/2} for Schedule 1 and Schedule 2 was predicted to be 6.7 and 4.4 days, respectively, while bolus dose administration resulted in elevated protein levels for 1.9 days. Similar results were observed under 10 μg treatment. These simulations indicated that split-dose regimens may result in prolonged duration of elevated protein availability compared to bolus dose administration. While the greatest differences in Rmax and ΔT_{1/2} were observed between bolus dose and split-dose regimens, it is worth noting that differences were also seen between Schedule 1 and Schedule 2. Specifically, the Schedule 2 regimen, designed to mimic a “burst” release upon the initial administration or injection, resulted in a statistically significant increase in Rmax, but a decrease in predicted duration of elevated protein expression, ΔT_{1/2} when compared to the Schedule 1 regimen. Overall, the computational model described herein predicted prolonged duration of elevated protein resulting from split-dose administration as compared to bolus dose administration.

### Example 6: Sustained Delivery of mRNA Vaccine Elicits Improved Immune Response Compared to Bolus Dosing

To evaluate the potential immunological benefit of sustained administration for mRNA vaccines, a mouse immunogenicity study that compared split versus bolus dose regimens was conducted.

The immunogenicity studies included split-dosed groups of BALB/c mice, (10 mice/dose group, aged 8-9 weeks) that were immunized via intradermal injection with 20 μL/quad of mRNA Vaccine 2, made according to the procedure outlined in Example 1, at days 0, 2, 4, 7 and 9. The mice then received a single injection of 50 μL/quad of mRNA Vaccine 2 at day 28. Bolus dosed groups of 10 BALB/c mice, aged 8-9 weeks, were immunized via intradermal injection with 50 μL/quad with mRNA Vaccine 2 at day 0 and day 28. Blood draws for serological assays were performed on days 21 and 42.

Antibody binding titers against pre-fusion RSV-F protein were evaluated using an ELISA following a previously described protocol (Zhang, L. et al, Vaccine, 2018, 36 (52), 8119-8130). Three hundred and eighty four well ELISA plates were coated with 2 μg/mL of purified recombinant prefusion RSV-F protein and incubated overnight at 4 C (McLellan, J. S. et al., J. Virol. 2011, 85, (15), 7788-96). Plates were then washed and blocked using 3% milk in PBS-T for 90 minutes at room temperature. Mouse sera were serially diluted in blocking buffer, transferred to the coated plates and incubated at room temperature for 2 hours. Plates were then washed 6× with PBS-T. After washing, HRP conjugated goat anti-mouse antibody (Thermo Fisher Scientific) diluted at 1:10000 in 3% milk in PBS-T was added to the plates and plates were incubated at room temperature for 1 hour. Plates were washed again 6× with PBS-T and developed with SuperBlu TMB solution (Virolabs). After 4 minutes the reaction was stopped, and absorbance was read at 450 nm using an EnVision microplate reader (Perkin Elmer). Endpoint titers were defined as the reciprocal of the end point dilution where the serum sample had an optical density signal greater than or equal to 2.5× the background.

10 BALB/c mice (aged 8-9 weeks) were immunized intradermally with 20 μL/quad intramuscular injections of mRNA Vaccine 2 on day 0, day 2, day 4, day 7, and day 9, wherein a total of 0.1 and 0.5 μg of mRNA delivered over the split dose schedule. BALB/c mice (10 mice, aged 8-9 weeks) were immunized intradermally with either 0.1 or 0.5 μg of mRNA Vaccine 2 following the split-dose regimen of injections on day 0, day 2, day 4, day 7, and day 9 or with a single bolus injection of mRNA Vaccine 2. Both split-dose and bolus-dose groups received a second immunization at day 28 using a single bolus injection, as shown in FIG. 3A. Sera were collected on day 21 and day 42 and tested for binding to pre-fusion RSV F protein by ELISA as well as neutralization activity. The split dose regimen that included injections on day 0, day 2, day 4, day 7, and day 9 aimed to replicate antigen exposure that gradually reached a sustained release rate similar to what could be expected from a microneedle patch. As indicated by FIGS. 3B and 3C, RSV pre-F specific ELISA titers for both low (0.1 μg) and high (0.5 μg) split-dose vaccine groups were found to be highly immunogenic, as measured by high levels of serum antibody binding RSV pre-F protein when compared to a traditional bolus dose administration. The more pronounced improvement was seen in the low dose group, where split-dose administration of the vaccine resulted in an increase in ELISA titers by nearly 260× at day 21 (FIG. 3B; **p<0.01) relative to a bolus dose. ELISA titers for the 0.1 μg mRNA split dosing group were still superior to the bolus dose group following a booster dose at day 28, with a ˜170× improvement in response at day 42 (FIG. 3C; **p<0.01). The 0.1 μg mRNA split-dose group also showed comparable ELISA titers to the 0.5 μg mRNA bolus dose group at day 21, suggesting a dose sparing effect from sustained administration of mRNA/LNP vaccine. Further, neutralization titers for the 0.1 μg mRNA low dose group were improved when administered via split-dose regimen (FIG. 3D; **p<0.01). The 0.5 μg mRNA/LNP group also showed a statistically significant improvement in ELISA titers at day 21 when following the split-dose regimen. While day 42 split-dose ELISA and neutralization titers were not significantly improved for the high dose mRNA/LNP group, the titers did trend higher when compared to the bolus dose group.

### Example 7: Sustained Delivery of Subunit Protein Vaccine, in Conjunction with Adjuvant, Elicits Improved Immune Response Relative to Bolus Dosing

Sustained delivery of subunit protein vaccines has been previously investigated, with results generally showing an immunological benefit. This study was conducted to comprehensively evaluate the benefit of sustained delivery for mRNA vaccines and characterize how their responses compare to subunit protein vaccines, a mouse study to evaluate the humoral immune response of an unadjuvanted RSV subunit pre-F protein vaccine, DS-Cav1, following a split-dose of injections on day 0, day 2, day 4, day 7, and day 9 and bolus administration study design as was carried out for the mRNA Vaccine 2 vaccination study (FIG. 4A).

The immunogenicity studies included split-dosed groups (10 mice, aged 8-9 weeks) that were immunized via intradermal injection with mRNA Vaccine 2, as described in Example 1, at days 0, 2, 4, 7 and 9 (20 μL/quad) followed by a single immunization (50 μL/quad) at day 28. Bolus dosed groups (10 mice, aged 8-9 weeks) were immunized via intradermal injection (50 μL dose quad) with mRNA Vaccine 2 at day 0 and day 28. Blood draws for serological assays were performed on days 21 and 42. The control vaccine included an unadjuvanted RSV subunit pre-F protein vaccine, DS-Cav1, (hereinafter “RSV Subunit Vaccine 1”).

As more robust enhancement in antibody titers with a split-dose was observed at 0.1 μg vs. 0.5 μg groups for mRNA Vaccine 2, the RSV Subunit Vaccine 1 split dosing comparison was performed at the 0.1 μg dose level. In contrast to immunization with mRNA Vaccine 2, immunization with 0.1 μg of the RSV Subunit Vaccine 1 did not yield an improvement in elicited antibody response when administered via a split dose regimen. Split dose administration of the RSV Subunit Vaccine 1 at 0.1 μg showed inferior immune responses relative to a bolus dose, with ELISA titers at least 50-fold lower on day 21 and day 42 (FIGS. 4B, 4C; *p<0.05, ** p<0.01). Additionally, an improvement in neutralizing antibody titers of the RSV Subunit Vaccine was not observed for the 0.1 μg split dose group (FIG. 4D).

Subunit proteins are known to be weakly immunogenic when administered on their own and benefit from the inclusion of adjuvants to help stimulate the immune system. Some adjuvants (e.g. alum) are also known to serve as depots and alter immune response kinetics. To determine if an adjuvant would stimulate an immune response, a follow-up split-dose versus bolus-dose mouse immunogenicity study that profiled the humoral immune response of the DS-Cav1 subunit protein formulated with an LNP (hereinafter “RSV Subunit Vaccine 2”) was conducted. In this study, the split-dosing regimen used in the RSV Subunit Vaccine 1 study was maintained, which recapitulated another possible antigen delivery profile expected from a controlled release microneedle patch technology (FIG. 4A). An increase in antibody titers (˜18×) was observed after 0.1 μg of RSV Subunit Vaccine 2 was administered over a 9-day period (with injections on day 0, day 2, day 4, day 7, and day 9) when compared to a bolus administration of RSV Subunit Vaccine 2 (FIG. 5A; *p<0.05). The observed titers for the 0.1 μg split-dose RSV Subunit Vaccine 2 group were also comparable to the 0.5 μg titers from the bolus dose RSV Subunit Vaccine 2 group. By day 42, following a single bolus boost at day 28, the split dose group had significantly greater titers than the bolus dose group (FIG. 5B; **p<0.01). Neutralizing antibody titers were also significantly improved at 0.1 μg when comparing split and bolus dose groups (FIG. 5C; *p<0.05). Overall, these data suggest that the inclusion of an adjuvant may be necessary to realize an improvement in immunogenicity with sustained delivery of subunit proteins.

To complete the comparison of vaccine modalities and further inform on the utility of controlled release technologies for mRNA vaccines, antibody responses of RSV Subunit Vaccine 1 following the split dosing design used in the RSV Subunit Vaccine 2 were profiled (FIG. 4A). Consistent with findings of the first immunization study, the RSV pre-F mRNA/LNP vaccine showed improved ELISA titers when delivered over a 9-day period (with injections on day 0, day 2, day 4, day 7, and day 9). Specifically, the low dose (0.1 μg) group showed a ˜75× and ˜500-fold improvement in antibody titers at days 21 and 42, respectively, when compared to the 0.1 μg bolus dose group (FIG. 6A, B; **p<0.01). The 0.1 μg split-dose group also showed comparable titers to the 0.5 μg bolus group at days 21 and 42, further suggesting sustained administration of the mRNA/LNP vaccine could result in a dose sparing opportunity. Mice that received the 0.5 μg dose administered over 9 days (with injections on day 0, day 2, day 4, day 7, and day 9) showed a ˜3× improvement in antibody response at day 21 when compared to mice that received the same dose with a single bolus injection (FIG. 6A; **p<0.01). By day 42, the 0.5 μg split-dose and bolus dose groups had comparable titers. Neutralizing antibody titers were not improved for both the 0.1 or 0.5 μg groups (FIG. 6B). The observed lack of neutralizing antibody improvement from subunit vaccine experiments suggests that, unlike the regimen used in FIG. 3A, split-dose regimen for a subunit protein vaccine may result in inferior functional antibody generation and further highlights the importance of profiling and optimizing antigen release kinetics. Analysis of antibody titers achieved by RSV Subunit Vaccine 2, mRNA Vaccine 1, and mRNA Vaccine 2 tested in this study revealed that both modalities were able to elicit robust humoral immune responses; however, the mRNA/LNP vaccine showed greater improvement in immunogenicity with sustained delivery compared to bolus injection (˜500× vs ˜18×) than the adjuvanted subunit protein.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method of treating a disease or disorder in a patient in need thereof comprising:
providing an active pharmaceutical ingredient (API) to said patient comprising:
(a) administering a first split-dose of said API;
(b) waiting for a pre-determined amount of time to pass;
(c) administering an additional split-dose of said API; and
(d) optionally repeating steps (b) and (c);
wherein each split-dose comprises an amount of API that is less than the amount of said API that is determined to be therapeutically effective at treating said disease or disorder via a bolus dose.

2. The method of claim 1, wherein the API is provided to the patient as an mRNA composition comprising an mRNA encoding an antigen and a pharmaceutically acceptable carrier.

3. The method of claim 2, wherein the mRNA composition further comprises a lipid nanoparticle (LNP).

4. The method of claim 3, wherein the LNP comprises a cationic lipid, a phospholipid, cholesterol, and a poly(ethyleneglycol) lipid (“PEG-lipid”).

5. The method of claim 4, wherein the LNP comprises 30-65 mole % cationic lipid, 5-30 mole % phospholipid, 10-40 mole % cholesterol, and 0.5-4 mole % PEG-lipid.

6. The method of claim 3, wherein the LNP comprises 55-65 mole % cationic lipid, 5-15 mole % phospholipid, 25-35 mole % cholesterol, and 1-2.5 mole % PEG-lipid.

7. The method of claim 6, wherein the phospholipid is distearoylphosphatidylcholine (“DSPC”) and the cationic lipid is (13Z, 16Z)-N, N-dimethyl-3-nonyldocosa 13, 16-dien-1-amine.

8. The method of claim 4, wherein the LNP comprises 5-15 mole % DSPC, 25-35 mole % cholesterol, 1-2.5 mole % ePEG2000-DMG, and 55-65 mole % (13Z, 16Z)-N, N-dimethyl-3-nonyldocosa 13, 16-dien-1-amine.

9. The method of claim 1, wherein the total amount of API provided to the patient by administration of all split-doses is equal to X % of the amount of the API provided in a bolus dose of said API, wherein X is less than or equal to 100.

10. The method of claim 1, wherein the amount of API in each split-dose is the same.

11. The method of claim 1, wherein the amount of API in each split-dose is not the same.

12. The method of claim 1, wherein the therapeutic effect is the same or greater than such effect when said API is provided to the patient as a bolus dose.

13. The method of claim 1, wherein the duration of time reflecting elevated protein expression (“ΔT_{1/2}”) of the API provided as a split-dose is greater than the ΔT_{1/2} when the API is provided as a bolus dose.

14. The method of claim 1, wherein the ΔT_{1/2} of the API provided as a split-dose is at least 2-10 times greater than the ΔT_{1/2} when the API is provided as a bolus dose.

15. The method of claim 1, wherein the maximum plasma API concentration in the area under the curve (“Rₘₐₓ”) of the API provided as a split-dose is less than the Rₘₐₓ when the API is provided as a bolus dose.

16. The method of claim 1, wherein the maximum plasma API concentration in the area under the curve (“Rₘₐₓ”) of the API provided as a split-dose is at least 50% less than the Rₘₐₓ when the API is provided as a bolus dose.

17. The method of claim 1, wherein the area under the curve (“AUC”) of the API provided as a split-dose is approximately the same as the AUC when the API is provided as a bolus dose.

18. A method of inducing an immune response in a patient in need thereof comprising:
providing an active pharmaceutical ingredient (API) to said patient comprising:
(a) administering a first split-dose of said API;
(b) waiting for a pre-determined amount of time to pass;
(c) administering an additional split-dose of said API; and
(d) optionally repeating steps (b) and (c);
wherein each split-dose comprises an amount of API that is less than the amount of said API that is determined to be therapeutically effective at inducing an immune response via a bolus dose.

19. The method of claim 18, wherein the total amount of API provided to the patient by administration of all split-doses is equal to X % of the amount of the API provided in a bolus dose of said API, wherein X is less than or equal to 100.

20. The method of claim 18, wherein the maximum plasma API concentration in the area under the curve (“Rₘₐₓ”) of the API provided as a split-dose is less than the Rₘₐₓ when the API is provided as a bolus dose.


SEQUENCE LISTING (2 sequences)

SEQ ID NO: 1 - RNA, 1521 nt, synthetic construct
  source 1..1521: mol_type = other RNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 2 - RNA, 1725 nt, synthetic construct
  source 1..1725: mol_type = other RNA
atggaactgctgattctcaaagccaacgccatcaccactatactgaccgccgtgaccttctgctttgcctcgggccagaacatcaccgaagaattttaccagtcaacctgttccgccgtgtcgaagggttacttgagcgccctgagaactggatggtacacgagcgtcatcaccatcgagctctcgaacatcaaggagaacaagtgcaacgggaccgatgccaaggtcaagctgatcaagcaggaactggacaagtacaagaacgccgtgactgagctccagcttctcatgcaatccacccccgccactaacaacagagcccggcgcgagctgcctaggttcatgaactacactctgaacaacgcgaagaaaaccaacgtgaccctctccaaaaagcgcaagcgccggttcctcggatttctgctgggcgtgggaagcgcgattgcctccggcgtggcagtgtccaaggtcctgcacctggaaggagaagtcaacaagatcaagagcgcccttctttccaccaacaaggcagtggtgtcactttcgaacggggtgtccgtgctgacctctaaggtcttggacctcaagaactacattgacaagcagctgctgcccatcgtcaacaagcagtcctgctccatctccaacatcgaaaccgtcatcgagttccagcagaagaacaatcggctgctggaaattaccagagagttcagcgtgaatgcgggagtgaccactcccgtgtccacttacatgctcactaacagcgagctgttgtccctgattaacgacatgccaatcaccaacgatcagaagaagctcatgtccaacaacgtccaaattgtgcggcagcagtcttactccatcatgtcaatcattaaggaagaggtgctggcatacgtggtgcagctgccgctctacggagtgattgacaccccgtgctggaagctgcacaccagccctctctgtaccactaataccaaggagggatccaacatctgtctgacgcgcaccgacaggggttggtactgcgacaacgctggctcggtgtccttcttcccgcaagcggagacttgcaaagtgcagagcaatcgcgtgttttgcgacaccatgaactccctgactctgcctagcgaagtgaacctgtgcaacgtggacattttcaacccgaaatacgactgcaagatcatgacgtcaaagactgatgtgtcatcctccgtgatcacatcactcggcgctattgtgtcctgttacggaaagaccaagtgcaccgcctccaacaagaaccggggaatcatcaagactttctccaatggctgcgactatgtgtccaacaagggcgtggacaccgtgtccgtcggcaacactctgtattacgtgaacaagcaggaagggaagtcgctctacgtcaagggcgaacccatcatcaacttctacgatccgctggtgttcccgtccgacgagttcgatgccagcatttcccaagtcaacgaaaagatcaaccagtccctcgccttcattcggaaatcggatgaactgctgcataacgtgaacgcgggaaagtcaaccaccaacatcatgatcactaccatcatcattgtgatcatcgtgatcctgctttcgctgatcgcggtcggtctgttgctgtactgcaaagctcggagcactcccgtgacactgagcaaggatcaactgtccgggattaacaacattgccttctcgaattga